(19) United States
(12) Patent Application Publication
(10) Pub. No.: US 2013/0085106 A1
(43) Pub. Date: Apr. 4, 2013

(54) METHODS AND FORMULATIONS FOR OXYTOCIN TREATMENT OF SUBSTANCE USE, PSYCHIATRIC AND OTHER DISORDERS

(71) Applicants: Cort A. Pedersen, Durham, NC (US); Marla L. Boccia, Charlotte, NC (US); Alexei Kampov-Polevoi, Chapel Hill, NC (US)

(72) Inventors: Cort A. Pedersen, Durham, NC (US); Marla L. Boccia, Charlotte, NC (US); Alexei Kampov-Polevoi, Chapel Hill, NC (US)

(73) Assignee: THE UNIVERSITY OF NORTH CAROLINA AT CHAPEL HILL, CHAPEL HILL, NC (US)

(21) Appl. No.: 13/698,947

(22) PCT Filed: May 19, 2011
(86) PCT No.: PCT/US11/37169

§ 371 Date: Dec. 19, 2012

Related U.S. Application Data
(60) Provisional application No. 61/346,347, filed on May 19, 2010.

(51) Int. Cl. A61K 38/11

(52) U.S. Cl. CPC A61K 38/11
USPC 514/11.6

(57) ABSTRACT

The invention provides a method of treating a psychiatric or medical disorder in a mammalian subject, the method comprising administering to the subject an effective amount of an oxytocin receptor agonist (e.g., oxytocin). Optionally, the oxytocin receptor agonist is delivered by intranasal administration. Further provided is a method of increasing social cognition, increasing social functioning, increasing empathy, increasing trust of others, reducing paranoia and/or reducing hostility in a subject with a psychotic disorder, a mood disorder characterized by psychotic features, a personality disorder or a pervasive developmental disorder, the method comprising administering to the subject an effective amount of an oxytocin receptor agonist. The invention also contemplates a method of preventing opioid dependence, opioid tolerance and/or opioid withdrawal symptoms in a subject receiving opioid treatment for pain relief, the method comprising administering to the subject an effective amount of an oxytocin receptor agonist. The invention also provides pharmaceutical compositions comprising an effective amount of an oxytocin receptor agonist and a pharmaceutically acceptable carrier.

### STATEMENT OF PRIORITY

This application is a 35 U.S.C. §371 national phase application of International Application Serial No. PCT/US2011/037169, filed May 19, 2011, which claims the benefit of U.S. Provisional Application No. 61/346,347; filed May 19, 2010, the disclosures of which are incorporated by reference in their entireties.

### RELATED APPLICATION INFORMATION

This application claims the benefit of U.S. Provisional Application No. 61/346,347; filed May 19, 2010, the disclosure of which is incorporated by reference herein in its entirety.

### STATEMENT OF FEDERAL SUPPORT

This invention was supported in part by funding provided under Grant No. RR025747 from the North Carolina Translational and Clinical Sciences Institute (NC TRaCS). The United States government has certain rights in this invention.

### FIELD OF THE INVENTION

The invention relates to methods and formulations for treating substance use, psychiatric and other disorders; in particular, the invention relates to pharmaceutical formulations and methods of administering an oxytocin receptor agonist for the treatment of substance use, psychiatric and other disorders.

### BACKGROUND OF THE INVENTION

Oxytocin has been reported to exert a large number of effects in the body and the brain. The classic physiological effects of oxytocin in humans as well as all placental mammals are uterine contractions and milk ejection. These are conclusively known to occur only under the hormonal conditions at the end of pregnancy and postpartum when oxytocin receptors proliferate in the uterus and mammary tissue and lactogenesis occurs. Oxytocin is approved by the United States Food and Drug Administration (FDA) for induction or facilitation of labor and postpartum contraction of the uterus. Intranasal oxytocin spray is also available in other countries to facilitate nursing in new mothers.

Novartis manufactures intranasal oxytocin spray (SYNTOCINON® Spray) which is indicated for facilitating initiation of milk letdown when new mothers are starting to breast-feed. This product was FDA-approved for this indication in the United States from 1962-1997 and continues to be marketed in other countries. Marketing in the United States stopped after 1997 when Novartis let FDA approval lapse because prescriptions for this product had declined markedly in this country.

### Alcohol Dependence and Withdrawal:

Consumption of ethanol (the type of alcohol in beer, wine and liquor) on a daily basis or numerous times per week for sustained periods (more than 1-2 weeks), especially at high doses, usually results in physical and psychological dependence on alcohol. The symptoms of alcohol dependence are described in the fourth, revised edition of the Diagnostic and Statistic Manual (DSM-IV-R, developed and published by the American Psychiatric Association, which summarizes the criteria for diagnosing psychiatric disorders) and include the following: forming tolerance to alcohol, i.e., requiring increasing doses over time to get the same degree of effect, a number of behavioral symptoms and often the onset of withdrawal symptoms within 12-72 hours after dependent individuals abruptly cease or cut back significantly on consumption of ethanol. The specific symptoms of withdrawal include tremors, anxiety, nausea with or without vomiting, bouts of sweating (diaphoresis), agitation, disturbances in perception that are tactile, visual and/or auditory (ranging from mild to overt hallucinations), headache or feelings of tightness around the head and disorientation/confusion that can range from mild to full-blown delirium. These symptoms are often quantified using the Clinical Institute Withdrawal Assessments Scale for Alcohol (CIWA). Other symptoms include elevated blood pressure, heart rate and, sometimes, body temperature. In extreme cases, cardiovascular collapse with a precipitous drop in blood pressure can occur and can be fatal. Seizures and psychotic symptoms can also occur. In patients admitted to hospital for medical treatment for alcohol dependence, the standard of care is to measure withdrawal symptoms at short, regular intervals so worsening of symptoms is quickly detected and promptly treated as described below.

Tolerance involves neurochemical changes in the brain that are as yet not fully understood resulting in decreasing magnitude of the effects of drugs when they are repeatedly administered. Addictive drugs, including alcohol, have rewarding emotional and/or physical effects which increase motivation to repeatedly consume these drugs. Animals and addiction prone people who have unlimited access to these drugs increase the amount of drug taken over time to compensate for the diminishing effectiveness of the drug due to tolerance formation. Because of the neurochemical changes in the brain occurring during tolerance formation, distressing physical and emotional symptoms of withdrawal occur if, after a sustained period of regular administration, addictive drug intake is abruptly stopped or the dosage is significantly reduced. To avoid these withdrawal symptoms, addicted individuals will go to great lengths to continue to obtain and consume these drugs, a condition called dependence. If the supply of drug is disrupted and withdrawal symptoms develop, dependent (addicted) individuals' thoughts and actions become fixated on the drug and finding an alternative source of the drug, a behavioral and emotional state referred to as craving.

### Standard Treatment for Alcohol Withdrawal (Medical Detoxification):

In patients with no history of severe alcohol withdrawal (i.e. they have never had seizures, delirium tremens or psychotic symptoms after a sustained bout of drinking and have had no significant medical complications from chronic alcohol ingestion such as gastrointestinal bleeding or cirrhosis of the liver), PRN (when necessary, from the Latin pro ra nata) administration of one or a few doses of a benzodiazepine or barbiturate medication is usually sufficient to control elevations of withdrawal symptoms if they occur. PRN doses are given when withdrawal symptom measurements rise above specific values (there is no firm consensus on these values, they vary, although not widely, among treatment sites). The doses of the standard medications are gradually tapered during and stopped by the end of a 3-5 day period. Sometimes patients with no history of dangerous withdrawal symptoms or medical complications will not be admitted to an inpatient unit but, rather, undergo medical detoxification as outpatients. This typically involves patients coming to a clinic each day over a 3-5 day period for evaluation of their withdrawal symptoms. They are given a limited supply of medication each day to control withdrawal symptoms should they occur at home. The amount of medication given each day is gradually tapered during and stopped by the end of the treatment period. In patients with a history of prior severe or dangerous withdrawal symptoms or medical complications from chronic alcohol ingestion, benzodiazepine or barbiturate medications are given initially at higher doses and on a standing schedule (i.e., doses are given at multiple, evenly spaced time points during the day) as well as PRN doses for acute elevations in withdrawal symptoms. The doses of these medications and the frequency at which they are given are gradually diminished during and then stopped at the end of the treatment period which is typically 4-6 days in length.

A major drawback of current treatments for alcohol withdrawal is that benzodiazepines and barbiturates are in the same class of drugs as alcohol (sedative-hypnotics) and have similar effects on neurotransmitter systems in the brain. Therefore, benzodiazepine and barbiturate treatment tends to maintain high levels of tolerance to alcohol so following medical detoxification patients continue to experience prolonged psychological withdrawal that results in craving for alcohol and heightened risk for relapse as described below.

### Alcohol Craving and Relapse:

Individuals with alcohol dependence usually experience craving for alcohol, especially after medical detoxification from alcohol or during periods when they try to cut back on the amount of alcohol they consume. Craving consists of chronic or frequent intrusive thoughts (obsessions) about drinking, how much better the patient would feel if they drank and where to get a drink. Craving is usually accompanied by strong urges to drink. Dependent individuals usually have difficulty keeping craving obsessional thoughts out of their mind and controlling impulses to drink. Craving and urges to consume drink are exacerbated by the chronic anxiety and difficulty coping with stress alcohol dependent subjects experience for long periods of time after they stop drinking (weeks to months, well after they are no longer in danger of having dangerous withdrawal symptoms). Cravings and urges to drink often lead to relapse (resuming drinking). Resumption of drinking is reinforced because it decreases cravings and diminishes anxiety and, therefore, once they start drinking again alcohol dependent individuals find it very difficult to stop drinking. Because dependent individuals have high tolerance for alcohol, when they relapse they tend to drink heavily because large quantities of alcohol are necessary to control anxiety, cope with stress and suppress craving. Craving is measured using instruments such as the Obsessive Compulsive Drinking Scale and the Penn Alcohol Craving Scale and the onset and degree of relapse is determined by interviewing patients using instruments like Timeline Followback Instrument (TLFB, Sobell et al., Brit. J. Addict. 83:393-402 (1988)) about when they started to drink again and how much. Breathalyzer testing or blood alcohol concentration measurements are additional ways to detect relapse. Relapse is quantified in terms of number of days sober before patients start to drink again and the amount of alcohol consumed after drinking resumes.

### Standard Treatment of Alcohol Craving and Relapse:

The treatment approaches that are most effective in helping alcohol dependent individuals cope with alcohol cravings and resist relapse are social support groups such as Alcoholics Anonymous as well as individual and group psychotherapy. FDA-approved pharmacological treatments are much less effective and include disulfuram (Antabuse), naltrexone, a mu opioid receptor antagonist, and acamprosate, an NMDA glutamatergic receptor antagonist that increases GABA_{A} receptor activity.

### Opioid Dependence and Withdrawal:

Individuals who have consumed opioid prescription or street drugs (e.g., morphine, oxycodone, hydrocodone, hydromorphone, fentanyl, heroin) daily or numerous times per week for sustained periods (more than 1-2 weeks), especially at high doses, become physically and often psychologically dependent on these drugs. The symptoms of opioid dependence are described in the DSM-IV-R and include the following: requiring increasing doses of opioid drugs to obtain the same physical and/or psychological effects, a number of behavior changes, and usually the onset of specific withdrawal symptoms if consumption of opioid drugs is abruptly ceased or significantly reduced. The physical symptoms of withdrawal include dysphoric mood, muscle aches and cramps, diarrhea and abdominal pain due to contractions of the bowels, nausea and/or vomiting, shakes, sweating, pupillary dilation, piloerection (hairs standing on end), lacrimation (tear formation), rhinorrhea (secretions from the nose), yawning, fever, and insomnia. Symptoms vary in their number and severity depending on the degree of dependence. They can be excruciatingly unpleasant. Opioid withdrawal symptoms are often measured using instruments such as the Objective Opiate Withdrawal Scale (OOWS, Handelsman et al., Am J Drug Alcohol Abuse 13: 293-308 (1987)).

### Standard Treatments of Opioid Withdrawal:

If their history indicates that patients may be able to successfully abstain from opioid use, medical detoxification of opioid dependent patients can be achieved either by (a) gradual tapering and then stopping opioid doses (this can be done in the hospital or in the outpatient setting and is usually a lengthy process), (2) replacing the opioid drugs that are being abused with SUBOXONE® (a combination of the mixed opioid agonist buprenorphine and the opioid antagonist naltrexone) or methadone treatment, or (3) abruptly stopping opioid drug administration (this is almost always done in hospital) and administering on a standing and/or PRN basis medications that diminish the severity of withdrawal symptoms over a treatment period of approximately 4-7 days. The doses of these medications are gradually tapered over the course of and stopped at the end of the treatment course. During inpatient medical detoxification, withdrawal symptoms are evaluated at regular intervals, sometimes using an instrument like the OOWS, to determine if standing doses of medications to control symptoms are adequate and to detect intensification of withdrawal symptoms that require PRN dosing. In patients whose history indicates that they will not be able to tolerate abstinence, withdrawal symptoms are often controlled by substituting the opioid drugs that have been misused by opioid drugs, such as methadone or buprenorphine, that can be given and monitored at an outpatient clinic.

### Opioid Craving and Relapse:

Individuals with opioid dependence usually experience craving for opioid drugs, especially after medical detoxification or during periods when they try to cut back on the amount of opioid drugs they consume. Craving consists of chronic or frequent intrusive thoughts (obsessions) about opioid drugs, how much better the patient would feel if they could take those drugs and where to find them. Craving is usually accompanied by strong urges to consume. Dependent individuals usually have difficulty keeping craving obsessional thoughts out of their mind and controlling impulses to use. Craving and urges to consume opioid drugs are exacerbated by the chronic anxiety and difficulty coping with stress opioid dependent subjects experience for long periods of time after they stop consuming (weeks to months, well after they are no longer in danger of having dangerous withdrawal symptoms). Many opioid dependent individuals also suffer chronic pain of various types (treatment of these conditions with opioids is often how they became dependent) which intensifies after stopping opioid drugs. Cravings and urges to consume often lead to relapse. Resumption of consumption is reinforced because it decreases cravings and diminishes anxiety and, therefore, once they start consuming again opioid dependent individuals find it very difficult to stop consuming. Because dependent individuals have high tolerance for opioid drugs, when they relapse they tend to consume large amounts because these quantities are necessary to control anxiety, cope with stress and suppress craving. Craving is measured using instruments such as the Heroin Craving Questionnaire (Schuster et al., Exp Clin Psychopharmacol 3: 424-431 (1995); Heinz et al., J Subst Abuse Treat 31:355-364 (2006)) which can be modified for use with patients who use opioid drugs other than heroin. Structured interviews to complete instruments like the Timeline Followback Instrument (TLFB, Sobell et al., Brit. J. Addict. 83:393-402 (1988)) are used to determine when patients relapsed and how much they have taken on a daily basis. Urine drug screens are also used to detect relapse. Relapse is quantified in terms of number of days sober before patients start to consume opioid drugs again and the amount of those drugs taken after use resumes.

### Standard Treatment of Opioid Craving and Relapse:

The treatment approaches that are most effective in helping opioid dependent individuals cope with opioid cravings and resist relapse are social support groups such as Narcotics Anonymous as well as individual and group psychotherapy. In recent years, treatment with SUBOXONE® has proven to be very effective in suppressing opioid craving and illicit use of other opioid drugs. Currently, however, there are no effective non-opioid drug treatments to decrease opioid craving and relapse.

### Diagnosis of Schizophrenia:

The diagnostic criteria for schizophrenia are summarized in the DSM-IV-R. Hallmark symptoms include hallucinations, delusions and disorganized thinking.

### Social Dysfunction in Schizophrenia:

Social dysfunction is the most disabling consequence of schizophrenia and responds poorly to currently available antipsychotic medications. The loss of brain volume and decline in general mental abilities that occur in many individuals with schizophrenia appear to contribute substantially to social dysfunction. However, recent evidence indicates that aspects of mentation that facilitate social decisions and behavior are particularly impaired in schizophrenia. This type of cognition, “social cognition,” comprises emotion recognition (e.g., identifying the emotional states of others from their facial expressions and other social cues), attributional style (beliefs about the causes of events) and theory of mind (inferring the thoughts and feelings of others), areas that are consistently impaired in schizophrenia, and which are related to real-world outcomes, such as social and community functioning. In addition, social behavior in schizophrenia may also be compromised by paranoia; individuals with persecutory delusions act more socially distant and stand-offish than individuals without persecutory delusions.

### Standard Pharmacological Treatment of Schizophrenia:

All effective medications for schizophrenia block dopamine D2 receptors. Older medications whose mechanism of action is primarily D2 receptor blockade are referred to as typical antipsychotics. More recently drugs that are less potent and selective in blocking D2 receptors and that also block serotonin 5-HT2 receptors have been introduced. These “atypical” antipsychotic medications have not proven to be more effective than typical antipsychotics medications in decreasing hallucinations, delusions, disorganized thinking or social dysfunction but generally produce fewer side effects resulting from blocking D2 receptors in the nigrostriatal dopamine pathway in the brain (tremor, muscle contractions, etc.) and may decrease the risk of developing permanent involuntary muscle movements (tardive dyskinesia).

There is a need in the art for improved treatments for substance use, psychiatric and other behavior disorders.

### SUMMARY OF THE INVENTION

The present invention is based, in part, on the discovery that an oxytocin receptor agonist (e.g., oxytocin) can be administered to a subject to treat a range of psychiatric disorders and medical disorders (e.g., fibromyalgia and/or chronic fatigue syndrome). Intranasal administration is particularly advantageous as it is non-invasive and facilitates self-administration outside of a clinical setting. For example, an oxytocin receptor agonist can be administered intranasally, which deposits considerable oxytocin into the brain, to treat subjects with a substance use disorder so as to reduce a withdrawal symptom, tolerance, craving and/or reduce relapse.

One beneficial effect of treatment with an oxytocin receptor agonist as compared with conventional therapies for medical detoxification of subjects with alcohol or opioid dependence is that oxytocin receptor agonists do not reinforce high levels of drug tolerance. In contrast, conventional therapies (e.g., benzodiazepine and barbiturate treatment for alcohol dependency or methadone and buprenorphine treatment for opioid dependence) tend to maintain high levels of tolerance so that following medical detoxification patients continue to experience prolonged psychological withdrawal that results in craving and heightened risk for relapse. By reducing tolerance, treatment with an oxytocin receptor agonist may reduce craving and help subjects to stop drug consumption after relapse. In addition, among patients who require opioid treatment to control pain, co-administration of oxytocin may preserve the full analgesic effect of the initial opioid dose thereby preventing the need for acceleration of the opioid dose and decreasing the risk of the patient becoming addicted.

As another potential advantage, pharmaceutical formulations comprising an oxytocin receptor agonist according to the present invention may be less expensive than conventional agents used to prevent relapse of a dependence disorder (e.g., disulfuram, naltrexone and SUBOXONE®). Many patients cannot afford these conventional drug products, thereby increasing the risk of relapse.

As another nonlimiting advantage, an oxytocin receptor agonist can be administered to a mammalian (e.g., human) subject, to treat a subject with a psychotic disorder (e.g., schizophrenia) or a mood disorder with psychotic features. In contrast with conventional treatments (e.g., typical and/or atypical anti-psychotics), the methods of the invention may improve social cognition, and therefore, social function in these patients. This would be an important achievement because social dysfunction is the major cause of disability in patients with schizophrenia and schizoaffective disorder.

Accordingly, as a first aspect the invention provides a method of treating a psychiatric or medical disorder in a mammalian subject (e.g., a human subject) in need thereof, the method comprising administering to the subject an effective amount of an oxytocin receptor agonist (e.g., oxytocin).

As a further aspect, the invention provides a method of treating a psychiatric or medical disorder in a mammalian subject (e.g., a human subject) in need thereof, the method comprising intranasally administering to the subject an effective amount of an oxytocin receptor agonist (e.g., oxytocin).

In embodiments of the invention, the disorder is a dependence disorder, such as a substance use disorder.

In embodiments of the invention, the method is practiced to reduce tolerance, a withdrawal symptom, craving, relapse and/or antisocial behavior associated with substance dependence and/or withdrawal.

In embodiments of the invention, the disorder is a psychotic disorder or a mood disorder with psychotic features.

In embodiments of the invention, the method is practiced to reduce a psychotic symptom, increase social cognition, increase social functioning, increase empathy, reduce paranoia, increase trust of others and/or reduce hostility in a mammalian (e.g., human subject in need thereof.

In embodiments of the invention, the disorder is a disorder characterized by anxiety, fear, depression, pain and/or intolerance to stress (e.g., fibromyalgia and/or chronic fatigue syndrome).

In embodiments of the invention, the disorder is a disorder characterized by social dysfunction and/or lack of empathy.

In embodiments of the invention, the method is practiced to increase social function, increase social cognition and/or increase empathy in a mammalian subject (e.g., a human subject) in need thereof.

In representative embodiments, the method further comprises adjunct therapy.

In further representative embodiments, the method comprises identifying a subject as belonging to a particular population, e.g., a subject dependent on an addictive substance and/or activity, a subject experiencing withdrawal from an addictive substance and/or activity, a subject experiencing craving for an addictive substance and/or activity, a subject having high tolerance to an addictive substance and/or activity, a dependent (i.e., addicted) subject that has relapsed, a subject in the prodromal state of a psychiatric disorder, a subject experiencing a first episode of a psychiatric disorder and/or a subject having impaired social cognition, impaired social function, impaired empathy, impaired trust of others, increased paranoia and/or increased hostility.

As still another aspect, the invention provides a pharmaceutical composition comprising an effective amount of an oxytocin receptor agonist and a pharmaceutically acceptable carrier for the treatment of a psychiatric or medical disorder in a mammalian subject (e.g., a human subject).

As yet a further aspect, the invention provides a pharmaceutical composition for intranasal administration comprising an effective amount of an oxytocin receptor agonist and a pharmaceutically acceptable carrier for the treatment of a psychiatric or medical disorder in a mammalian subject (e.g., a human subject).

The invention also encompasses a method of increasing social cognition, increasing social functioning, increasing empathy, reducing paranoia, increasing trust of others, and/or reducing hostility in a mammalian subject (e.g., a human subject) with a psychotic disorder, a mood disorder characterized by psychotic features, a personality disorder or a pervasive developmental disorder, the method comprising administering to the subject an effective amount of an oxytocin receptor agonist. In representative embodiments, the method further comprises evaluating social cognition, social functioning, empathy, paranoia, trust of others and/or hostility in the subject before and/or after administration of the oxytocin receptor agonist. In further representative embodiments, the method comprises identifying a subject as having an impairment in social cognition, social function, empathy and/or trust of others and/or increased paranoia and/or hostility.

Further provided is a pharmaceutical composition comprising an effective amount of an oxytocin receptor agonist and a pharmaceutically acceptable carrier for increasing social cognition, social functioning, empathy and/or trust in others and/or reducing paranoia and/or hostility in a mammalian subject (e.g., a human subject). Optionally, the pharmaceutical composition is formulated for intranasal administration.

The invention also encompasses a method of preventing opioid dependence, opioid tolerance and/or opioid withdrawal symptoms in a mammalian subject receiving opioid treatment for pain relief in need thereof, the method comprising administering to the subject an effective amount of an oxytocin receptor agonist.

In representative embodiments, the subject is currently dependent on an addictive substance or has a history of such addiction. In representative embodiments, the subject is not currently dependent on an addictive substance or have a history of such addiction.

The invention also provides a pharmaceutical composition comprising an effective amount of an oxytocin receptor agonist and a pharmaceutically acceptable carrier for preventing opioid dependence, opioid tolerance and/or opioid withdrawal symptoms in a mammalian subject receiving opioid treatment for pain relief. Optionally, the pharmaceutical composition is formulated for intranasal administration.

These and other aspects of the invention are described in more detail in the following description of the invention.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

As used in the description of the invention and the appended claims, the singular forms “a,” “an” and “the” are intended to include the plural forms as well, unless the context clearly indicates otherwise.

The term “about,” as used herein when referring to a measurable value such as a dosage, time, temperature, and the like, is meant to encompass variations of ±20%, ±10%, ±5%, ±1%, ±0.5%, or even ±0.1% of the specified amount.

Numerical ranges as described herein are intended to be inclusive unless the context indicates otherwise. For example, the numerical range of “1 to 10” or “1-10” is intended to be inclusive of the values 1 and 10.

Also as used herein, “and/or” refers to and encompasses any and all possible combinations of one or more of the associated listed items, as well as the lack of combinations when interpreted in the alternative (“or”).

The features of the invention described herein can be used in any combination. Moreover, the present invention also contemplates that in some embodiments of the invention, any feature or combination of features set forth herein can be excluded or omitted.

By “consisting essentially of” as used herein, it is meant that the indicated compound, composition, formulation and the like does not include any other material elements (i.e., elements that materially impact the structure and/or function of the compound, composition or formulation).

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the onset of at least one clinical symptom, relapse and/or progression of the disease or disorder.

The terms “prevent,” “preventing” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the methods of the present invention.

An “effective” amount as used herein is an amount sufficient to achieve a desired outcome, and is optionally a treatment effective amount.

A “treatment effective” amount as used herein is an amount that is sufficient to treat (as defined herein) the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, the terms “increase,” “increases,” “increased” and “increasing” as well as “enhance,” “enhances,” “enhanced,” “enhancing,” and “enhancement” and similar terms, indicates an elevation in the specified parameter, for example, an elevation of at least about 10%, 20%, 30%, 40%, 50%, 2-fold, 3-fold, 4-fold, 5-fold, 10-fold, 15-fold, 20-fold or more. In representative embodiments, increases/enhancements in an indicated parameters are relative to a healthy control population without the relevant disorder or condition. In embodiments of the invention, increases/enhancements in the indicated parameter are relative to the subject's own condition prior to the methods of the invention.

As used herein, the terms “reduce,” “reduces,” “reduced,” “reducing,” “reduction” as well as “impair,” “impairs,” “impaired,” “impairing,” “impairment” and similar terms indicate a decrease in the specified parameter, e.g., of at least about 10%, 20%, 30%, 40%, 50%, 60%, 75%, 80%, 85%, 90%, 95%, 97% or more. In particular embodiments, the reduction or impairment results in no or essentially no (i.e., an insignificant amount, for example, less than about 10% or even 5%) detectable activity. In representative embodiments, reductions/impairments in an indicated parameter are relative to a healthy control population without the relevant disorder or condition. In embodiments of the invention, reductions/impairments in the indicated parameter are relative to the subject's own condition prior to the methods of the invention.

A “dependent” or “addicted” subject (and similar terms) as used herein is a subject that has physical and/or psychological dependence on a drug, substance or activity.

The present invention can be practiced for medical, veterinary and research purposes. Subjects to be treated by the methods of the invention include both avian and mammalian subjects, mammalian subjects including but not limited to humans, non-human primates (e.g., monkeys, baboons, and chimpanzees), dogs, cats, goats, horses, pigs, cattle, sheep, and the like, and laboratory animals (e.g., rats, mice, gerbils, hamsters, and the like). Avian subjects include chickens, ducks, turkeys, geese, quails and birds kept as pets (e.g., parakeets, parrots, macaws, and the like). Suitable subjects include both males and females and subjects of all ages including infant, juvenile, adolescent, adult and geriatric subjects.

In representative embodiments, the subject is in need of the methods of the invention, e.g., the subject has a psychiatric disorder or other disorder as described herein.

In representative embodiments, the subject is an animal model, e.g., an animal model for a psychiatric disorder or other disorder as described herein.

In embodiments of the invention, the subject does not have obsessive compulsive disorder.

In embodiments of the invention, the subject does not have autism.

In embodiments of the invention, the subject does not have an autism spectrum disorder.

In embodiments of the invention, the subject does not have schizophrenia.

In embodiments of the invention, the subject does not have a psychotic disorder.

In embodiments of the invention the subject is dependent on an addictive substance and/or activity and/or has a history of such addiction.

In embodiments of the invention the subject is not dependent on an addictive substance and/or activity and/or have a history of such addiction.

In embodiments of the invention, the subject cannot tolerate and/or does not respond to a satisfactory extent to a conventional treatment for the disorder. For example, the subject may be a subject with schizophrenia or a psychotic disorder and cannot tolerate and/or does not respond satisfactorily with typical and/or atypical anti-psychotic medications.

As another example, the subject may be an alcohol addicted subject that has undergone detoxification, but cannot tolerate and/or does not respond to a satisfactory extent to the conventional agents used to help maintain sobriety and/or prevent relapse (e.g., naltrexone, disulfuram and/or acamprosate).

As a further illustration, the subject is an opioid addicted subject undergoing detoxification that cannot tolerate and/or does not respond to a satisfactory extent to the agents conventionally used to reduce symptoms of opioid withdrawal (e.g., methadone, buprenorphine, SUBOXONE®, clonidine and/or an opioid antagonist [for example, naltrexone] and/or drugs to treat specific withdrawal symptoms such as nausea, vomiting, diarrhea, abdominal cramps, muscle cramps/twitches, muscle/bone pain, anxiety, and/or rhinorrhea/lactimation including without limitation lorazepam). In other embodiments, the subject is a subject that has undergone detoxification for opioid addiction, but cannot tolerate and/or does not respond to a satisfactory extent to the conventional agents used to help maintain sobriety and/or prevent relapse (e.g., SUBOXONE®).

In representative embodiments, the methods of the invention comprise identifying a subject as belonging to a particular population, e.g., a subject dependent on an addictive substance and/or activity, a subject experiencing withdrawal from an addictive substance and/or activity, a subject experiencing craving for an addictive substance and/or activity, a subject having high tolerance to an addictive substance and/or activity, a dependent (i.e., addicted) subject that has relapsed, a subject in the prodromal state of a psychiatric disorder, a subject experiencing a first episode of a psychiatric disorder, a subject having impaired social cognition, impaired social function, impaired empathy, impaired trust of others, increased paranoia, increased hostility and/or any other population described herein.

As one aspect, the invention provides a method of treating a psychiatric or medical disorder in a subject, the method comprising administering to the subject an effective amount of an oxytocin receptor agonist (e.g., oxytocin).

Further encompassed by the present invention is a method of treating a psychiatric or medical disorder in a subject, the method comprising intranasally administering to the subject an effective amount of an oxytocin receptor agonist (e.g., oxytocin).

The methods of the invention can be carried out with any suitable oxytocin receptor agonist including oxytocin and/or an oxytocin analog such as carbetocin (1-butanoic acid-2-(O-methyl-L-tyrosine)-1-carbaoxytocin; Hunter et al., Clin. Pharmacol. Ther. 52: 60-67 (1992)). A number of other oxytocin receptor agonists are known in the art and include peptide and non-peptide molecules. Nonlimiting examples of oxytocin receptor agonists include: 4-threonine-1-hydroxy-deaminooxytocin, 9-deamidooxytocin (an analog of oxytocin containing a glycine residue in place of the glycinamide residue; du Vigneuaud, J. Med. Chem. 9:55-57 (1966)), 4-deamido-oxytocin (an analog of oxytocin containing a glutamic acid residue in place of glutamine; Photaki and du Vigneaud, J. Am. Chem. Soc. 87: 908-913 (1965)), 7-D-proline-oxytocin and its deamino analog (du Vigneuaud, J. Am. Chem. Soc. 88: 3847-3850 (1966)), (2,4-diisoleucine)-oxytocin and its deamino oxytocin analog (Hruby et al., J. Med. Chem. 13:185-187 (1970); Urry et al., Proc. Natl. Acad. Sci. USA 66: 111-116 (1970)), 1-desamino-1-monocarba-E12-Tyr(OMe)-oxytocin (Veznik et al., Am. J. Vet. Res. 4): 425-429 (1979); Cort et al., Am. J. Vet. Res. 43: 1283-1285 (1982); Cort et al., Am. J. Vet. Res. 40:430-432 (1979)), [Thr4-Gly7]-oxytocin (Chadio and Antoni, J. Mol. Endocrinol. 10: 107-114 (1993)), oxypressin, deamino-6-carba-oxytoxin (Krejci et al, Regul. Pept. 2: 285-291 (1981)), 4-β-alanine-oxytocin (Manning and du Vigneaud, Biochemistry 4: 1884-1888 (1965)), Ile-conopressin, atosiban, desmopressin, and 1-deamino-oxytocin in which the disulfide bridge between residues 1 and 6 is replaced by a thioether.

Oxytocin receptor agonists include biologically active fragments of oxytocin. For example, oxytocin fragment 4-9 has been reported to be more potent than the full-length molecule (see, e.g., Burbach et al., Eur. J. Pharmacol. 94: 125-131 (1983)). By “biologically active” is meant that the fragment substantially retains (e.g., at least about 50%, 60%, 70%, 80%, 90%, 95% or more) at least one biological activity of full-length oxytocin, e.g., with respect to reducing a withdrawal symptom(s), craving, tolerance and/or relapse in a dependent subject and/or reducing psychosis and/or increasing social cognition in a subject with a psychotic disorder (e.g., schizophrenia) or a mood disorder with psychotic features.

Other useful forms of oxytocin or other oxytocin receptor agonists include pharmaceutically acceptable active salts of oxytocin or other oxytocin receptor agonists as well as active isomers, enantiomers, polymorphs, solvates, hydrates and/or prodrugs of the same.

Any psychiatric disorder now known or later identified can be treated according to the present invention (see, e.g., DSM-IV-R for a comprehensive listing of psychiatric disorders).

In embodiments of the invention, the disorder is a psychiatric disorder including but not limited to a substance use disorder (e.g., dependence on and/or withdrawal from alcohol, opioids, cocaine, cannabis, benzodiazepines, nicotine and/or amphetamines), a psychotic disorder (e.g., schizophrenia, schizoaffective disorder, delusional disorder, and the like), an anxiety disorder (including posttraumatic stress disorder, panic, generalized anxiety, a social anxiety disorder and/or agoraphobia), a pervasive developmental disorder characterized by social deficits (e.g., autistic disorder, Rett's disorder, childhood disintegrative disorder, Asperger's disorder, and/or pervasive developmental disorder not otherwise specified), a childhood attachment disorder (e.g., reactive attachment disorder, separation anxiety disorder and/or selective mutism) and/or a female reproductive state-related disorder (e.g., premenstrual dysphoric disorder and/or perimenopausal depression) and/or a personality disorder (e.g., paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and/or obsessive-compulsive personality disorder) and/or a medical disorder characterized by pain, anxiety and/or fatigue (e.g., fibromyalgia and/or chronic fatigue disorder).

Psychiatric disorders further include but are not limited to disorders of thinking and cognition such as schizophrenia and delirium; amnestic disorders; disorders of mood, such as affective disorders and anxiety disorders (including post-traumatic stress disorder, separation anxiety disorder, selective mutism, reactive attachment disorder, stereotypic movement disorder, panic disorders, agoraphobia, specific phobias, social phobia, obsessive-compulsive disorder, acute stress disorder, generalized anxiety disorder, substance-induced anxiety disorder and/or anxiety disorder not otherwise specified); disorders of social behavior; disorders of learning and memory, such as learning disorders (e.g., dyslexia); motor skills disorders; communication disorders (e.g., stuttering); pervasive developmental disorders (e.g., autistic disorder, Rett's disorder, childhood disintegrative disorder, Asperger's disorder, and/or pervasive developmental disorder not otherwise specified) and dementia; depressive disorders (including major depressive disorder, dysthmyic disorder, depressive disorder not otherwise specified, postpartum depression); seasonal affective disorder; mania; bipolar disorders (including bipolar I disorder, bipolar II disorder, cyclothymic disorder, bipolar disorder not otherwise specified); attention-deficit and disruptive behavior disorders (including attention deficit disorder with hyperactivity disorder, conduct disorder, oppositional defiant disorder and/or disruptive behavior disorder not otherwise specified); drug addiction/substance use disorders (e.g., withdrawal from and/or dependence on a substance including without limitation opiates, amphetamines, alcohol, hallucinogens, benzodiazepines, cannabis, inhalants, phencyclidine, sedatives, hypnotics, anxyolytics and/or cocaine); alcohol-induced disorders; amphetamine-induced disorders; caffeine-induced disorders; cannabis-induced disorders; cocaine-induced disorders; hallucinogen-induced disorders; inhalant-induced disorders; nicotine-induced disorders; opioid-induced disorders; phencyclidine-induced disorders; sedative, hypnotic or anxyolytic-induced disorders; agitation; apathy; psychoses; irritability; disinhibition; schizophreniform disorder; schizoaffective disorder; delusional disorder; brief psychotic disorder, shared psychotic disorder; substance-induced psychotic disorder; psychotic disorder not otherwise specified; unipolar disorders, mood disorders (e.g., mood disorder with psychotic features); somatoform disorders; factitious disorders; disassociative disorders; eating disorders such as anorexia nervosa, bulimia nervosa and/or eating disorder not otherwise specified; sleeping disorders (e.g., dyssomnias such as primary insomnia, primary hypersomnia, narcolepsy, breathing-related sleep disorder and circadian rhythm sleep disorder and/or parasomnias); impulse control disorders (e.g., kleptomania, pyromania, trichotillomania, pathological gambling and/or intermittent explosive disorder); sexual disorders (e.g., hypoactive sexual desire disorder, female sexual arousal disorder, male erectile disorder, female orgasmic disorder, male orgasmic disorder and/or and sexual disorder not otherwise specified); adjustment disorders; personality disorders (e.g., paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and/or obsessive-compulsive personality disorder); Tic disorders (e.g., Tourette's disorder, chronic motor or vocal tic disorder, transient tic disorder and/or tic disorder not otherwise specified); and any combination of the foregoing as well as any other disorder or group of disorders described in the DSM-IV-R.

In representative embodiments, the disorder is a dependence disorder including without limitation a substance use disorder, gambling disorder, eating disorder (e.g., overeating) and/or a sexual addiction. A dependence disorder can involve any drug, substance or activity that results in physical and/or psychological dependence in the user. Likewise, substance use disorders can involve any drug or substance that results in physical and/or psychological dependence in the user. For example, physical dependence can result in withdrawal symptoms (e.g., tremors) and/or craving when the drug is withdrawn. Nonlimiting examples of drug substances include: alcohol; cocaine and cocaine derivatives; a cannabinoid (e.g., cannabis and/or hashish; active ingredient delta-9-tetrahydrocannabinol [THC]); a depressant such as a barbiturate (e.g., AMYTAL®, NEMBUTAL®, SECONAL®, PHENOBARBITAL®), a benzodiazepine (e.g., ATIVAN®, HALCION®, LIBRIUM®, VALIUM®, XANAX®), flunitrazepam, gamma-hydroxybutyrate and methaqualone; a dissociative anesthetic such as ketamine, phencyclidine (PCP) and phencyclidine derivatives; hallucinogens such as lysergic acid diethylamide (LSD), mescaline and psilocybin; opioids and opium derivatives such as heroin, morphine and morphine derivatives, thebaine, hydromorphone, oxycodone, hydrocodone, codeine, meperidine, and pentazocine; stimulants such as cocaine and cocaine derivatives, amphetamines, methylenedioxy-methamphetamine (MDMA), methamphetamine, methcathinone, methylphenidate and nicotine and nicotine derivatives; and fentanyl and fentanyl derivatives; and any combination of the foregoing.

Accordingly, in particular embodiments, the invention is practiced to treat a dependence disorder such as a substance use disorder (e.g., alcohol dependence, opioid dependence, nicotine dependence, cocaine dependence, benzodiazepine dependence), a gambling disorder, an eating disorder and/or a sexual addiction.

The invention can be used to treat any aspect of a dependence disorder, for example, to reduce tolerance to a drug substance, to reduce a withdrawal symptom, to reduce craving, to reduce relapse and/or to reduce antisocial behavior associated with substance dependence and/or withdrawal.

Tolerance refers to the decreasing magnitude of the effects of the substance or activity to which dependence has developed after repeated use and appears to be the result of neurochemical changes in the brain. Thus, the addicted subject compensates by increasing the dosage/frequency of the addictive drug or activity over time to maintain the same effect.

As used herein “reduce tolerance” (and similar terms) indicates that the subject's sensitivity to the drug or activity of addiction is enhanced, i.e., the subject will experience the physical and psychological effects of the drug or activity at lower levels of consumption as compared with the level of tolerance in the absence of the methods of the invention and/or the subject will experience reduced craving for the addictive drug or activity and/or a longer duration before relapse and/or between relapses.

As used herein “reduce a withdrawal symptom” (and similar terms) indicates that there is a reduction in the frequency and/or intensity of one or more withdrawal symptoms experienced by the subject and/or a reduction in the number of episodes of withdrawal symptoms requiring pharmaceutical intervention (e.g., with conventional pharmaceutical therapy such as benzodiazepines and/or barbituates for alcohol withdrawal) and/or a reduction in the total amount of the pharmaceutical intervention required to treat the subject undergoing withdrawal and/or the rate of taper of such pharmaceutical intervention can be more rapid as compared with the level that would be experienced in the absence of the methods of the invention. In one study for example, remarkably the inventors observed that four of six patients undergoing medical detoxification for alcohol addiction required no pharmacologic intervention (in this study, with lorazepam; Example 2 in the working examples).

Withdrawal symptoms can be physical and/or emotional and occur after the drug or activity to which the subject has developed dependence is stopped or significantly reduced. Withdrawal symptoms are addressed extensively herein. For example, withdrawal symptoms associated with alcohol withdrawal include without limitation tremors, chills, anxiety, nausea with or without vomiting, bouts of sweating (diaphoresis), agitation, disturbances in perception that are tactile, visual and/or auditory (ranging from mild to overt hallucinations), headache or feelings of tightness around the head, disorientation/confusion that can range from mild to full-blown delirium, elevated blood pressure, elevated heart rate, elevated body temperature, cardiovascular collapse with a drop in blood pressure (in extreme cases), seizures and/or psychotic symptoms. Numerous methods and instruments for measuring withdrawal symptoms are known in the art. One instrument for quantifying withdrawal symptoms from alcohol is the Clinical Institute Withdrawal Assessment for Alcohol Scale (CIWA-Ar).

Withdrawal symptoms associated with opioid withdrawal include without limitation dysphoric mood, muscle aches and cramps, diarrhea and abdominal pain due to contractions of the bowels, nausea and/or vomiting, shakes, sweating, pupillary dilation, piloerection, lacrimation, rhinorrhea, yawning, fever and/or insomnia. Methods for measuring withdrawal symptoms from opioids are known in the art and include, for example, instruments such as the Objective Opiate Withdrawal Scale (OOWS) and the Subjective Opiate Withdrawal Scale (SOWS).

In humans, “craving” consists of chronic or frequent intrusive thoughts (obsessions) about the drug/activity of addiction, how much better the subject would feel if s/he took the drug or engaged in the activity, and how and where s/he can get access to the drug or activity. These obsessive thoughts are usually accompanied by strong urges to consume the addictive drug or engage in the addictive activity. Substance dependent animals also exhibit drug seeking behavior.

As used herein “reduce craving” (and similar terms) refers to a decrease in the frequency and/or intensity of craving during withdrawal from the drug/activity of addiction and/or afterwards as the subject attempts to remain sober and avoid relapse and/or a decrease in the number of episodes of craving requiring pharmaceutical intervention (e.g., with conventional pharmaceutical therapy such as disulfuram, naltrexone, acamprosate for alcohol craving or buprenorphine and/or naltrexone for opioid craving) and/or a decrease in the total amount of the pharmaceutical intervention required to treat the subject experiencing craving as compared with the level that would be observed in the absence of the methods of the invention.

Numerous methods and instruments for measuring craving are known in the art. Exemplary instruments for quantifying craving for alcohol include the Obsessive Compulsive Drinking Scale and the Penn Alcohol Craving Scale. Craving for opioids can be measured using instruments such as the Heroin Craving Questionnaire, which can be modified for use with subjects who use opioid drugs other than heroin. One hundred millimeter visual analog scales can also be used to assess how much the subject “wants,” “needs,” and “craves” the substance or activity of addiction.

Relapse refers to the subject's restarting use of the drug of addiction or participation in the addictive activity, which can be temporary or of longer duration. As used herein, the term “reduce relapse” and similar terms refer to an increase in the duration of time (e.g., days, weeks, months, years) the subject refrains from the addictive drug or activity and/or an increase in duration of time between relapses and/or a decrease in the length and/or intensity (e.g., amount of addictive drug consumed or participation in addictive activity) of each relapse.

In humans, the onset and degree of relapse can be determined by any method known in the art, e.g., by interviewing subjects using instruments such as the Timeline Followback Instrument (TLFB). Objective tests such as breathalyzer testing (for alcohol), blood and/or urine testing can also be used to detect relapse. Relapse can be quantified in terms of the number of days sober and/or the amount of the drug consumed after drug consumption resumes. Similar measures can be used to assess relapse from addictive activities. Relapse can be followed for any suitable time period, e.g., over days, weeks, months or even years. In embodiments of the invention, the rate of relapse is reduced by the methods of the invention over a period of about one month, six months, 12 months, 18 months or even 24 months.

In embodiments of the invention, the methods of the invention result in an increased compliance with psychotherapy (e.g., increased attendance) and/or increased participation in social support groups.

In embodiments of the invention, the psychiatric disorder is a psychotic disorder or mood disorder with psychotic features. To illustrate, the psychotic or mood disorder can be schizophrenia, schizoaffective disorder, delusional disorder, a depressive disorder with psychotic features, and/or a bipolar disorder with psychotic features. Subjects with schizophrenia can be subdivided into different subtypes defined according to the most significant and predominant characteristics present in each person at each point in time (e.g., paranoid type, disorganized type, catatonic type, undifferentiated type and residual type). In embodiments of the invention, the subject is diagnosed with, or suspected of having, any of these subtypes of schizophrenia.

According to this aspect, the invention is optionally practiced to reduce a psychotic symptom, increase social cognition, increase social functioning, increase empathy, reduce paranoia, increase trust of others and/or reduce hostility in a subject with a psychotic disorder (e.g., a schizophrenic subject such as a subject with paranoid schizophrenic subject), a mood disorder with psychotic features, a personality disorder or a pervasive developmental disorder. In representative embodiments, the method further comprises evaluating one or more psychotic symptoms, social cognition, social functioning, empathy, paranoia, trust of others and/or hostility in the subject before and/or after administration of the oxytocin receptor agonist.

The term “social cognition” is understood in the art and includes, for example, emotion recognition (e.g., identifying the emotional states of others from their facial expression and/or other social cues), attributional style (e.g., beliefs about the causes of events) and/or theory of mind (e.g., inferring the thoughts and feelings of others).

Methods of assessing psychotic symptoms, social cognition, social functioning, paranoia, trust of others and/or hostility are well-known in the art and illustrative examples are described herein, for example, the total and item scores on the Positive and Negative Symptom Scale (PANSS), the Paranoia Scale, and instruments to measure social cognition/function and empathy (Brüne Theory of Mind Picture Stories Task, the Trustworthiness Task and the Face Emotion Identification Task [FEIT], Emotion Recognition-40 Task [ER-40], Ambiguous Intentions Hostility Questionnaire [AIHQ], Reading the Mind in the Eyes test, Interpersonal Reactivity Index of empathy (IRI), Specific Levels of Functioning Scale). Role-playing tasks can also be used to assess social competence (e.g., conversational skills, ability to perceive distress in others). The Specific Levels of Functioning Scale (SLOF), a questionnaire that quantifies social and other functional domains can be completed by the subject and an informant familiar with the subject for an extended period of time, is another suitable instrument for assessing this population. Particular PANSS item scores related to social function include P6 (suspiciousness/persecution [paranoia]), P7 (hostility), N4 (passive/apathetic social withdrawal), G8 (uncooperativeness) and G16 (active social avoidance). See also, discussion of different instruments in Example 9 in the working examples.

In representative embodiments, the disorder is a disorder characterized by anxiety, fear, depression, pain and/or intolerance to stress. For example, the disorder can be an anxiety disorder (including posttraumatic stress disorder), a depressive disorder, and/or a premenstrual dysphoric disorder. According to this embodiment, the invention can also be used to treat a medical disorder such as fibromyalgia and/or chronic fatigue syndrome.

According to this aspect, the invention is optionally practiced to reduce anxiety, fear, depression, pain and/or intolerance to stress in a subject with a disorder characterized by anxiety, fear, depression, pain and/or intolerance to stress such as an anxiety disorder, a depressive disorder, a premenstrual disorder, fibromyalgia and/or chronic fatigue syndrome. Methods and instruments to assess anxiety, fear, depression, pain and stress tolerance are known in the art.

The invention also provides methods of treating a disorder characterized by social dysfunction. Examples of such disorders include without limitation a personality disorder, a pervasive developmental disorder and/or a disorder of social behavior.

According to this aspect, the invention is optionally practiced to increase social function, increase social cognition and/or empathy in a subject with a disorder characterized by social dysfunction. Methods and instruments to assess social function, social cognition and/or empathy are known in the art, for example, as described herein (e.g., see discussion above regarding psychotic disorders and the working examples).

In embodiments of the invention, the subject is a subject that is in the prodromal period prior to the onset of a psychotic disorder (such as schizophrenia or schizoaffective disorder), and the subject is administered an effective amount of an oxytocin receptor agonist to reduce the likelihood of progression to the full-blown disease, delay the onset of the disease, and/or reduce the severity of the disease (e.g., to prevent the disease)). In the case of schizophrenia, the prodromal period can be marked by the onset of disorganized thinking and/or speech patterns, unusual thoughts, beliefs and/or perceptions including quasi-hallucinations and quasi-delusions that do not yet meet the criteria for schizophrenia (e.g., are not yet sufficiently pervasive and/or severe). In particular embodiments, the invention is practiced to treat a subject in the prodromal phase of schizophrenia to reduce and/or delay the decline in social cognition and/or social function and/or psychotic episodes that occur early in the course of the disease (e.g., to prevent schizophrenia, to prevent the decline in social cognition and/or social function and/or, to prevent psychotic episodes).

In other embodiments of the invention, the subject is a subject that is experiencing the first episode of a psychotic disorder and/or a mood disorder with psychotic features, and the subject is administered an effective amount of an oxytocin receptor agonist to reduce the severity and/or duration of one or more symptoms associated with the disorder, to reduce the likelihood and/or severity of subsequent episodes (i.e., relapse) and/or to delay the onset of subsequent episodes (e.g., to prevent the disorder). In particular embodiments, the subject is experiencing a first episode of schizophrenia, and the invention is practiced to reduce and/or delay the increase in number and/or intensity of psychotic symptoms as well as the decline in social cognition and/or social function that are typical of the disease (e.g., to prevent schizophrenia, to prevent psychotic symptoms and/or to prevent the decline in social cognition and/or social function). By “first episode” is meant a period of at least about one month during which the subject for the first time shows symptoms that meet criteria for active schizophrenia with the possible exception of the requirement that symptoms persist for six or more months. If the first episode occurs during a period less than about six months in duration in which the subject initially exhibits prodromal symptoms before and/or residual symptoms after the one month or longer active phase, a diagnosis of schizophreniform disorder is made. Alternatively, if the active phase is the only initial manifestation of the disorder, it is diagnosed as a schizophreniform disorder for the first six months. If the one month or longer active phase occurs six or more months after the onset of prodromal symptoms or the active phase without a prior prodromal stage persists for six or more months, a diagnosis of schizophrenia is made.

The present invention also finds use in preventing dependence, tolerance and/or withdrawal symptoms in subjects administered opioids for pain relief. Subjects that are treated with opioids for pain relief face the risk of developing opioid dependence (it is difficult to determine a priori which subjects are addiction prone). Further, as opioid tolerance develops, increasing dosages of opioids may have to be administered to maintain efficacy. In addition, if dependence does develop, the subject can experience withdrawal symptoms once opioid treatment is stopped. Administration of oxytocin in subjects administered opioids for pain relief can reduce the likelihood of dependence and/or tolerance (with a resulting need for increasing dosages to control pain).

The subject can be any subject (e.g., a mammalian or human subject) being administered an opioid for pain relief. In embodiments of the invention, the subject is currently dependent on a substance (e.g., alcohol, cocaine and/or any other addictive substance as described herein) or have a history of such dependence. These subjects may be particularly at high risk for developing opioid dependence and/or tolerance. In embodiments of the invention, the dependence is/was not opioid dependence. In embodiments of the invention, the subject is not currently dependent on a substance and does not have a history of dependence. Optionally, subject is receiving opioid treatment for pain control for a prolonged period of time (e.g., at least about 1, 2, 3, 4 or 6 weeks or 2, 3, 4, 6, 9 or 12 months or longer).

According to representative embodiments, the likelihood of developing opioid dependence is reduced, the likelihood of developing opioid tolerance is reduced, less total opioid is required to maintain pain control over time, the likelihood of needing to increase the dosage of the opioid to maintain pain relief is reduced, the opioid can be tapered off more rapidly and/or subjects may experience fewer and/or less severe withdrawal symptoms after the cessation of opioid administration as compared with the effects of opioid treatment for pain control in the absence of the methods of the invention.

Any suitable dosage of the oxytocin receptor agonist can be administered to give the desired response. Dosages of pharmaceutically active compounds can be determined by methods known in the art, see, e.g., Remington's Pharmaceutical Sciences (Maack Publishing Co., Easton, Pa). In particular embodiments, the dosage of the oxytocin receptor agonist ranges in potency from at least about 0.05, 0.1, 0.5, 1, 2, 3, 4, 5, 7, 10, 15, 20, 25, 30, 40, 50 international units (IU) of oxytocin and/or less than about 20, 25, 30, 40, 50, 75, 100, 150, 200, 250, 300, 500 or 1000 IU of oxytocin for a typical (e.g., 70 kg) human subject (including any combination of the lower and upper dosages as long as the lower value is less than the upper value). In particular embodiments, the dosage is from about 1 to about 100 IU, optionally from about 4 to about 25 or 50 IU.

Treatment can be short-term (e.g., acute; for hours or days) or can be a long-term, chronic regimen (e.g., weeks, months or years). In some instances, the treatment is a maintenance regimen that lasts for months, years or even the life of the subject.

For example, with respect to substance use disorders, relatively short-term treatment can be used to treat (reduce) withdrawal symptoms (e.g., for 2, 3, 4, 5, 6, 7, 8, 9 or 10 days or any range therein). For example, withdrawal periods for alcohol are typically about 3-5 days and for opioids about 4-7 days. To treat (reduce) craving and/or relapse, long-term treatment can be used, e.g., weeks, months, years, life of the patient, optionally on an as-needed basis.

An exemplary dosage scheme is administration from about 1, 2, 3, 4, 5 or 6 to about 2, 3, 4, 5, 10, 15 or 20 times a day (including any combination of the lower and upper values as long as the lower value is less than the upper value). In other embodiments, dosing is done once about every two days, every three days, every four days, every five days, every six days, once a week, every two weeks or once a month. In embodiments of the invention, dosing is on an as-needed basis (e.g., based on exacerbation of symptoms, craving, anxiety, and the like). Those skilled in the art will appreciate that for acute indications (e.g., medical detoxification), dosing will generally be one or more times per day. Further, for dependence disorders, the dosage and/or frequency of dosing typically tapers off over time.

In some embodiments, dosing is on an as-needed basis.

It is specifically intended that the invention encompasses any combination of dosing frequency and dosage amounts described herein.

The methods of the invention can further comprise the use of one or more adjunct therapies. In other embodiments, administration of the oxytocin receptor agonist is the sole pharmacologic treatment for the disorder. Examples of adjunct therapy include without limitation psychotherapy, participation in a social support group, and/or therapy with a pharmaceutical agent(s).

With respect to dependence disorders, exemplary adjunct therapy can include psychotherapy and/or participation in a social support group. For alcohol withdrawal, further adjunct therapy can include without limitation: treatment with a sedative-hypnotic drug (e.g., a benzodiazepine such as lorazepam and/or a barbiturate), aversion therapy (e.g., with disulfuram [Antabuse]), a mu opioid receptor antagonist (e.g., naltrexone), and/or a NMDA glutamatergic receptor antagonist (e.g., acamprosate). When treating opioid dependence, the adjunct therapy can include without limitation: treatment with an opioid agonist (e.g., buprenorphine, methadone) and/or combination with an opioid antagonist (e.g., naltrexone). Other therapies can be administered to treat specific withdrawal symptoms, e.g., an anti-emetic for nausea and/or vomiting (e.g., promethazine), a substance to treat diarrhea (e.g., loperamide), abdominal cramps (e.g., dicyclomine), muscle cramps/twitches (e.g., cyclobenzaprine), muscle/bone pain (e.g., acetaminophen, ibuprofen), anxiety (e.g., lorazepam), and/or rhinorrhea/lactimation (e.g., diphenhydramine). Clonidine can also be given as an adjunct therapy.

When treating a psychotic disorder such as schizophrenia, adjunct therapy can include without limitation treatment with a typical anti-psychotic (i.e., a dopamine D2 receptor blocker) and/or an atypical anti-psychotic (i.e., dopamine D2 receptor antagonist and serotonin 5-HT2 antagonist activity). Typical anti-psychotics include without limitation: Loxitane (loxapine), MELLARIL® (thioridazine), MOBAN® (molindone), NAVANE® (thiothixene), ORAP® (pimozide), PROLIXIN® (fluphenazine), STELAZINE® (trifluoperazine), THORAZINE® (chlorpromazine), and Trilafon (perphenazine). Atypical anti-psychotics include without limitation: ABILIFY® (apripiprazole), CLOZARIL® (clozapine), GEODON® (ziprasidone), INVEGA® (paliperidone), RISPERDAL® (risperidone), SEROQUEL® (quetiapine), SYMBYAX® (olanzpine [ZYPREXA®] plus fluxetine [PROZAC®]), and ZYPREXA® (olanzapine).

For treating impairments in social cognition and/or social function, adjunct therapy can include typical and/or atypical antipsychotic drugs (if the underlying disorder involves psychosis, such as schizophrenia) and/or training and/or psychotherapy for social cognition and/or social function.

The oxytocin receptor agonist (and any additional active compounds used as adjunct therapy) can be administered by any suitable route, including without limitation oral, rectal, transmucosal, intranasal, inhalation (e.g., via an aerosol), buccal (e.g., sublingual), vaginal, intrathecal, intraocular, transdermal, parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like.

With respect to intranasal delivery, any suitable method of intranasal administration can be employed. In particular embodiments, intranasal administration is by inhalation (e.g., using an inhaler, atomizer or nebulizer device), alternatively, by spray, tube, catheter, syringe, dropper, packtail, pledget, and the like. As a further illustration, the oxytocin receptor agonist can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by nasal drops, spray or aerosol. As used herein, aerosols can be used to deliver powders, liquids or dispersions (solids in liquid).

Many devices are known in the art for nasal delivery. Exemplary devices include particle dispersion devices, bidirectional devices, and devices that use chip-based ink-jet technologies. ViaNase (Kurve Technologies, Inc., USA) uses controlled particle dispersion technology (e.g., an integrated nebulizer and particle dispersion chamber apparatus, for example, as described in International patent publication WO 2005/023335). Optinose and Optimist (OptiNose, AS, Norway) and DirectHaler (Direct-Haler A/S, Denmark) are examples of bidirectional nasal delivery devices. Ink-jet dispensers are described in U.S. Pat. No. 6,325,475 (MicroFab Technologies, Inc., USA) and use microdrops of drugs on a millimeter sized chip. Devices that rely on iontophoresis/phonophoresis/electrotransport are also known, as described in U.S. Pat. No. 6,410,046 (Intrabrain International NV, Curacao, AN). These devices comprise an electrode with an attached drug reservoir that is inserted into the nose. Iontophoresis, electrotransport or phonophoresis with or without chemical permeation enhancers can be used to deliver the drug to the target region (e.g., olfactory).

Nasal delivery devices are also described in U.S. Pat. No. 6,715,485 (OptiNose AS); U.S. Pat. No. 6,325,475 (Microfab Technologies, Inc.); U.S. Pat. No. 6,948,492 (University of Kentucky Research Foundation); U.S. Pat. No. 6,244,573 (LyteSyde, LLC); U.S. Pat. No. 6,234,459 (LyteSyde, LLC); U.S. Pat. No. 6,244,573 (LyteSyde, LLC); U.S. Pat. No. 6,113,078 (LyteSyde, LLC); U.S. Pat. No. 6,669,176 (LyteSyde, LLC); U.S. Pat. No. 5,724,965 (Respironics Inc.); and U.S. Patent Publications US2004/0112378 A1; US 2004/0112379 A1; US 2004/0149289 A1; US 2004/0112380 A1; US 2004/0182388 A1; US 2005/0028812 A1; US 2005/0235992 A1; US 2005/0072430 A1 and US 2005/0061324 A1.

In embodiments of the invention, the oxytocin receptor agonist is administered as a slow-release depot, e.g., that is implanted subcutaneously.

In representative embodiments of the invention, the oxytocin receptor agonist (and/or any other active compound used as adjunct therapy) is administered to the central nervous system (CNS), for example, to the brain. As non-limiting examples, the active compound(s) can be administered to the spinal cord, brainstem (e.g., medulla oblongata, pons), cerebellum, midbrain (e.g., substantia nigra, ventral tegmental area, raphe nuclei, inferior colliculus), hypothalamus, thalamus, pituitary gland, pineal gland, limbic system (e.g., amygdala, hippocampus, septum, medial preoptic area), basal ganglia e.g., corpus striatum, globus pallidus), and cerebral cortex (e.g., occipital, temporal, parietal and frontal lobes). The active compound(s) can also be delivered into the cerebrospinal fluid (e.g., by lumbar puncture) for more disperse administration. The active compound(s) can be administered intravascularly to the CNS in situations in which the blood-brain barrier has been perturbed (e.g., brain tumor or cerebral infarct).

The active compound(s) can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery. In particular embodiments, the active compound(s) is administered in a liquid formulation by direct injection (e.g., stereotactic injection) to the desired region or compartment in the CNS. In other embodiments, the active compound(s) can be provided to the CNS by topical application to the desired region or by intra-nasal administration (e.g., for delivery to the brain).

The invention further contemplates a pharmaceutical composition comprising an effective amount of an oxytocin receptor agonist and a pharmaceutically acceptable carrier for the treatment of a psychiatric or medical disorder in a subject.

Also provided is a pharmaceutical composition for administration comprising an effective amount of an oxytocin receptor agonist and a pharmaceutically acceptable carrier for increasing social cognition and/or social functioning in a subject.

By “pharmaceutically acceptable” it is meant a material that is not toxic or otherwise undesirable.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

For injection, the carrier will typically be a liquid. For other methods of administration, the carrier may be either solid or liquid. For inhalation administration, the carrier will be respirable, and is typically in a solid or liquid particulate form.

The concentration of the active compound(s) in the pharmaceutical formulations can vary widely, e.g., from less than about 0.01% or 0.1% up to at least about 2% to as much as 20% to 50% or more by weight, and will be selected primarily by fluid volumes, viscosities, etc., in accordance with the particular mode of administration selected.

The active compound(s) can be formulated for administration in a pharmaceutical carrier in accordance with known techniques. See, e.g., Remington, The Science And Practice of Pharmacy (21^{st} Ed. 2005). In the manufacture of a pharmaceutical composition according to the invention, the active compound(s) (including physiologically acceptable salts thereof) is typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. One or more active compounds can be incorporated in the formulations of the invention, which can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

In particular embodiments, the pharmaceutical composition is administered to a mucosal surface, e.g., by intranasal, inhalation, intratracheal, oral, buccal, rectal, vaginal or intra-ocular administration, and the like.

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

It will be understood by those skilled in the art that the choice of suitable carriers, absorption enhancers, humectants, adhesives, etc., will typically depend on the nature of the active compound and the particular nasal formulation, for example, a nasal solution (e.g., for use as drops, spray or aerosol), a nasal suspension, a nasal ointment, a nasal gel, or another nasal formulation.

Optionally, drug solubilizers can be included in the pharmaceutical composition for intranasal administration to improve the solubility of the compound and/or to reduce the likelihood of disruption of nasal membranes which can be caused by application of other substances, for example, lipophilic odorants. Suitable solubilizers include but are not limited to amorphous mixtures of cyclodextrin derivatives such as hydroxypropylcylodextrins (see, for example, Pitha et al., (1988) Life Sciences 43:493-502).

Moist and highly vascularized membranes can facilitate rapid absorption; consequently, the pharmaceutical composition for intranasal administration can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition for intranasal administration can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

The pharmaceutical composition for intranasal administration can optionally contain an odorant, e.g., as described in EP 0 504 263 B1 to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

In representative embodiments, the pharmaceutical composition is formulated to comprise one or more of propyl-4-hydroxybenzoate, methyl-4-hydroxybenzoate and hemihydrated chlorobutanol in addition to the oxytocin receptor agonist.

In embodiments of the invention, the pharmaceutical composition is SYNTOCININ® Spray (Novartis).

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the active compound(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules containing the active compound(s), optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical formulations suitable for buccal (sub-lingual) administration include lozenges comprising the compound(s) in a flavored base, usually sucrose and acacia or tragacanth; and pastilles comprising the compound(s) in an inert base such as gelatin and glycerin or sucrose and acacia.

Pharmaceutical formulations suitable for parenteral administration can comprise sterile aqueous and non-aqueous injection solutions of the active compound, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are preferably presented as unit dose suppositories. These can be prepared by admixing the active compound(s) with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of an optionally buffered aqueous solution of the compound(s). Suitable formulations can comprise citrate or bis\tris buffer (pH 6) or ethanol/water and can contain from 0.1 to 0.2M active ingredient.

Having described the present invention, the same will be explained in greater detail in the following examples, which are included herein for illustration purposes only, and which are not intended to be limiting to the invention.

### Example 1

### Formulation of Oxytocin for Intranasal Administration

Oxytocin intranasal spray (SYNTOCINON® Spray) has been marketed internationally by Novartis Pharmaceuticals for several decades with the indication of assisting nursing mothers initiate adequate milk let down during the early postpartum period. In addition to oxytocin, SYNTOCINON® Spray contains additives that enhance absorption across epithelium as well as preservatives to prolong shelf life. The concentration of oxytocin in SYNTOCINON® Spray is 40 International Units (about 80 micrograms)/milliliter. SYNTOCINON® Spray is not currently marketed in the United States. However, approval of Investigational New Drug (IND) applications to use SYNTOCINON® Spray obtained from overseas pharmacies for specific new human research or clinical purposes can be obtained from the United States Food and Drug Administration.

### Example 2

### Intranasal Administration of Oxytocin as a Treatment for Alcohol Withdrawal

### Subjects:

Up to 80 alcohol dependent patients, 18-65 years of age, admitted to the University of North Carolina (UNC) Hospitals Clinical and Translational Research Center (CTRC), Family Practice or Hospitalist Service Units for medical detoxification are studied. There is no selection on the basis of gender, ethnicity or race.

### Inclusion/Exclusion Criteria:

### Inclusion Criteria:

1) At least one prior episode 2 days or longer in duration during which the subject experienced withdrawal symptoms that caused significant incapacitation (e.g., unable to work, do normal activities) OR at least one prior inpatient or outpatient medical detoxification during which the subject exhibited withdrawal symptoms of sufficient magnitude that sedative-hypnotic or anticonvulsant medication was required at least once on 2 consecutive days after cessation of or reduction in the use of alcohol following 2 weeks or more of heavy daily consumption; 2) Heavy daily consumption of alcoholic drinks: Beer-6-12 12 oz bottles/cans; or 5-9 16 oz bottles/cans; or 2-3.5 40 oz bottles/cans. Liquor-½-1¼ of a pint; or ⅜-¾ of a fifth; or ¼-½ of a 750 ml bottle. Wine-1.5-3 750 ml bottles for at least 2 weeks prior to the current admission (depending on history of withdrawal and rate of consumption the Principal Investigator may use discretion on these exact numbers); 3) age 18-65.

### Exclusion Criteria:

1) Current delirium, disorientation to place or person, seizures, acute or unstable psychosis, mania, dependence on substances other than alcohol, nicotine, caffeine or cannibis (abuse of other substances is not exclusionary), suicidal or homicidal ideation with strong intent, plans or recent attempt (ideation alone is not exclusionary); amnesia, dementia, cognitive impairment, significant neurological symptoms, compromising medical conditions (e.g., AIDS [HIV infection alone as well as adequately controlled conditions such as diabetes, hypertension, asthma will not be exclusionary], seizure disorder, emphysema, cancer); low body weight (BMI<17); history of anorexia nervosa or bulimia in the past 2 years; significant trauma, self injurious behavior or surgery in the previous 2 months; pregnancy; breast-feeding, parturition or breast-feeding in the past 6 months; 2) history of alcohol withdrawal-related seizures, delirium tremens or hallucinations, 3) current or past alcohol-related medical complications such as cirrhosis of the liver, esophageal varices, pancreatitis, severe gastritis, hemoptysis, hematochezia or melena, 4) ingestion during the 2 weeks prior to this admission of much more alcohol/day than during previous drinking binges that preceded the onset of withdrawal symptoms OR daily ingestion of significantly more than the maximum amount allowed in the inclusion criteria OR a blood alcohol level upon admission >300 mg/dl, 5) treatment/ingestion in the 72 hr prior to enrollment in the study and receiving the first intranasal dose of test substance with/of long half-life benzodiazepines or sedative hypnotic drugs other than short half-life benzodiazepines, 6) chronic treatment with benzodiazepines, barbiturates, anticonvulsants or stimulants, 7) well-documented history of inadequately treated baseline hypertension or tachycardia (SBP>150 or DBP>100 or P>110), 8) vital signs on admission or following admission but prior to recruitment into the study are SBP>190, DBP>125 or P>140 (indicates patients require standing benzodiazepine dosing or loading which disqualifies them from participation in the study), 9) any of the following laboratory values on admission: AST>165 U/L (normal range 19-55), alkaline phosphatase >378 U/L (normal range 38-126), non-fasting glucose >250 mg/ml (normal range 65-179), hematocrit <38% (normal range 41-53), hemoglobin <12 g/dl (normal range 13.5-17.5) or any other laboratory value that is outside the normal range including electrolytes or bilirubin or EKG abnormalities (mild-moderate elevations or glucose levels in diabetics will not be exclusionary), 10) inability to read well enough to complete study questionnaires (this will be determined by whether the prospective subject can read and understand the consent form without help). 11). Participation in any clinical trial within the past 60 days. 12). Court-mandated participation in alcohol treatment or pending incarceration. Concurrent depressive and/or anxiety disorders without psychotic features and psychotic disorders well-controlled on a stable (>1 month) regimen of antipsychotic medications are not exclusionary. Standing or medications for non-exclusionary medical disorders as well as psychiatric medications other than those excluded above are permitted.

These inclusion and exclusion criteria select subjects who are likely to experience withdrawal symptoms but are at low risk of developing dangerous withdrawal problems such as seizures, delirium tremens, Wernicke's encephalopathy or hallucinations and have no serious or unstable comorbidities. This is a population of alcohol dependent patients in whom symptom-triggered medication treatment has been shown to be as safe and results in less total medication administration as compared with fixed regimen benzodiazepine tapers (Mayo-Smith, JAMA 278: 144-151 (1997); Daeppen et al., Arch. Intern. Med. 162: 1093-1094 (2002); Lange-Asschenfeldt et al., Eur. Addict. Res. 9: 1-7 (2003)).

### Study Design, Methods and Procedures

### Study Design:

This is a double-blind, placebo controlled comparison of the efficacy of twice daily intranasal administration of oxytocin and placebo (saline) in reducing alcohol withdrawal symptoms, the number of bouts of withdrawal during which symptoms are of sufficient magnitude requiring lorazepam treatment (see definitions below) and the amount of lorazepam required to resolve withdrawal bouts. Treatment group assignment are random within each sex. Subjects are patients admitted to UNC Hospitals or the UNC CTRC for medical detoxification. Each treatment group is composed of up to 40 subjects. The study statistician constructs a randomization plan to randomize 80 subjects to 2 groups of 40 subjects each with a blocksize of 4. This is a permuted blocksize as this is a blind study.

### Procedures:

Some patients who are admitted to the UNC Hospitals Family Practice or Hospitalist Services for medical detoxification from alcohol come in through the UNC Hospitals Emergency Department during the late evening or early morning. Physical examination, medical and psychiatric history and lab tests (including CBC, electrolytes including magnesium, Na, K, BUN, creatinine, glucose, albumin, AST, alkaline phosphatase, GGT, TSH, B12, folate, blood alcohol concentration, urinalysis, urine toxicology screen and pregnancy test) as well as vital signs are routinely obtained by the Emergency Department or inpatient medical units during the admission process to UNC Hospitals. Physical examination, medical and psychiatric history and labs are obtained during an outpatient screening evaluation within a few days before admission to the UNC CTRC. If this evaluation determines that a subject does not qualify for the study but he/she is requesting medical detoxification, then the research staff endeavors to have the patient medically evaluated and treated. The manner in which this is done depends on when exclusionary information is obtained from the evaluation process. If this information becomes available while the subject is still in the CTRC outpatient clinic, research staff, with the subject's permission, contacts and then walks or transports the subject to the UNC Hospitals Emergency Department. If exclusionary information (e.g., lab values) is obtained after the subject has left the outpatient clinic, the subject is contacted as soon as possible by phone and given information about medical facilities where he/she can be evaluated for and receive medical detoxification. The CTRC does not have the capacity to safely conduct medical detoxification of subjects with complicated alcohol withdrawal history (DTs, seizures) or unstable medical conditions.

After admission to the UNC Hospitals medical units or CTRC, patients have their vital signs (VSs) measured and their withdrawal symptoms quantified using the revised Clinical Institute Withdrawal Assessment for Alcohol scale (CIWA-Ar scale, Sullivan et al., Brit. J. Addict. 84: 1353-1357 (1989)).

Informed consent is obtained the morning after admission to the UNC Hospitals medical units or before the outpatient screening evaluation before admission to the UNC CTRC. After consent is obtained, subjects are randomly assigned to a treatment group (oxytocin or placebo). Prior to receiving their first intranasal treatment, an ECG is obtained and blood drawn to obtain serum for assay of cytokine and allopregnanolone concentrations. Subjects also rate their symptoms using the Alcohol Withdrawal Symptom Checklist (AWSC, Pittman et al., Alcohol Clin. Exp. Res. 31: 612-618 (2007)) prior to receiving their first test dose. These procedures are done/overseen by research nurses or physicians. Subjects usually receive their first intranasal test dose between 1000 and 1200 hours, which consists of 6 insufflations of SYNTOCINON® Spray (approximately 24 IU) or placebo, with each insufflation given 30 seconds apart and alternating between nostrils. Test doses taken again at 1700 hr later that day (Admission Day 1) and at 0900 and 1700 hr on Admission Days 2 and 3. Oxytocin test treatments are administered from 5 ml intranasal spray vials designed to deliver 0.1 ml metered volume per insufflation. The placebo treatments are administered from 60 ml intranasal spray vials (each containing 30 ml of solution) also designed to deliver 0.1 ml metered volume per insufflation. Vials containing oxytocin and placebo spray are blind labeled by the UNC Investigational Drug Service. Research nurses or physicians oversee subject self-administration of all intranasal test doses. After enrollment on Admission day 1 and in the morning on Admission days 2 and 3, subjects are given multiple copies of the AWSC questionnaire and requested to complete one each time the unit nurses do CIWA ratings. These questionnaires are retrieved by research staff the following morning. Shortly after the morning test dose on Admission days 2 and 3, subjects complete the Alcohol Craving Visual Analog Scales (ACVAS), the Penn Alcohol Craving Scale (PACS), and the Profile of Mood States (McNair et al., (1971) EDITS manual for the profile of mood states. San Diego, Calif. Educational and Industrial Testing Service). In addition, a consumption diary is administered and given to the subject to quantify the amount of alcohol participants have been consuming prior to admission in the study. On Days 2 and 3, research nurses also draw (and subsequently process) blood serum samples to assay cytokine and allopregnanolone concentrations shortly after the morning test dose. Additional blood is drawn after the morning test dose on admission day 2 to obtain serum for tests to be run (magnesium, Cl, Ca, Na, K, BUN, creatinine, and GGT). Another ECG is also obtained shortly after the morning test treatment on Admission day 2. Research staff retrieves on a daily basis CIWA ratings and vital sign measurements obtained on each subject from their electronic medical record.

In all subjects, lorazepam doses are administered PO or IV (if subjects cannot take medication PO) whenever CIWA ratings are ≧12. CIWA ratings are obtained 1 hr after the initial dose of lorazepam and another dose administered if CIWA ratings ≧10. The standard lorazepam dose is 2 mg. Subjects may receive up to 8 repeated doses of lorazepam if their CIWA score remains between 10 and 20. If their CIWA score does not fall below 10 after 8 repeated doses then the subject is withdrawn from the study.

In the event a subject is moved to another unit during his/her hospital stay, the study continues as usual unless the move is due to medical complications that would require withdrawal from the study.

### Results:

Ten subjects were enrolled in this double blind study in which each subject was randomly assigned to receive either oxytocin (N=6) or placebo (N=4) by intranasal spray (6 insufflations/dose) twice daily for the first 3 days after stopping drinking during which they were inpatients on the research unit. Alcohol withdrawal symptoms were measured at least every 4 hours and every 1 hour after receiving each dose of lorazepam. Research nurses scored withdrawal symptoms using the CIWA scale modified to include measurements of blood pressure, heart rate, respiration rate, and body temperature. Subjects rated their own withdrawal symptoms using the AWSC. On study days 2 and 3, subjects also rated how much they craved alcohol using 2 instruments; the Penn Alcohol Craving Scale (PACS) and a 100 mm Alcohol Craving Visual Analog Scale (AWVAS; “not at all” on the left end of the 100 mm line and “extremely” at the right end) scored as the distance from the left end of the line where subjects put their mark.

The primary outcomes of this study were 1) total mgs of lorazepam required to control withdrawal symptoms during the 3 days of the protocol and 2) mean CIWA scores on study days 1, 2 and 3. Secondary outcomes were mean AWSC self-ratings on days 1, 2 and 3; PACS and ACVAS self ratings on days 2 and 3.

Of the 10 subjects in this sample, 8 were men and 2 were women. Oxytocin recipients included 5 men and 1 woman; placebo recipients included 3 men and 1 woman. All subjects successfully completed medical detoxification within the 3 days of the protocol. There were no unexpected adverse events.

Numerous significant differences were found using t test comparisons between treatment groups (see Table 1 below). This was particularly true for our primary outcome measures; total mg of lorazepam, mean CIWA scores on days 1, 2 and 3. The total mgs of lorazepam required to complete medical detoxification was much lower in the oxytocin treatment group. Quite surprisingly, 4 of the 6 subjects in this group required no lorazepam treatment at all. In contrast, all subjects in the placebo treatment group required multiple doses of lorazepam. Mean CIWA scores were also highly significantly lower on all 3 days of the protocol in the oxytocin compared to the placebo treatment group. After Bonferroni correction for multiple comparisons, the differences between treatment groups for all of our primary outcomes remained significant.

Several of our secondary outcome measures differed significantly between treatment groups. AWSC self-ratings of withdrawal symptoms were significantly lower in the oxytocin compared to the placebo treatment group on days 1 and 3 and approached significance on day 2. Self-ratings for alcohol craving on the PACS and ACVAS were significantly lower on day 2 in the oxytocin compared to the placebo treatment group. Craving measures on day 3 did not differ between treatment groups.

TABLE 1

| | Oxytocin | | Placebo | | | | |
|---|---|---|---|---|---|---|---|
| Measure | Mean | SD | Mean | SD | DF | t | p |
| Total lorazepam (mg) | 2.33 | ±4.08 | 16.50 | ±4.43 | 8 | 5.20 | .0008 |
| Mean CIWA Day 1 | 2.40 | ±2.23 | 11.11 | ±0.40 | 5.48 | 7.54 | .0004 |
| Mean CIWA Day 2 | 0.81 | ±2.25 | 5.38 | ±3.58 | 8 | 4.34 | .0025 |
| Mean CIWA Day 3 | 1.52 | ±0.82 | 2.34 | ±1.91 | 8 | 3.47 | .0085 |
| Mean AWSC Day 1 | 6.17 | ±1.25 | 16.30 | ±4.49 | 3.31 | 4.40 | .0177 |
| Mean AWSC Day 2 | 5.40 | ±2.58 | 20.6 | ±10.20 | 3.26 | 2.92 | .0557 |
| Mean AWSC Day 3 | 3.78 | ±2.17 | 9.55 | ±5.16 | 8 | 2.48 | .0379 |
| Mean PACS Day 2 | 13.33 | ±5.20 | 21.00 | ±4.55 | 8 | 2.39 | .0438 |
| Mean PACS Day 3 | 9.33 | ±8.21 | 12.75 | ±2.99 | 8 | 0.78 | .4553 |
| Mean ACVAS Day 2 | 33.83 | ±22.68 | 86.50 | ±14.54 | 8 | 4.04 | .0037 |
| Mean ACVAS Day 3 | 11.23 | ±13.22 | 38.93 | ±39.34 | 3.46 | 1.36 | .2564 |

SD = standard deviation; DF = degrees of freedom; lower values are related to
unequal variance in data from the two treatment groups; t = t value; p = significance
level (values ≦.05 [in bold] indicate significant differences between treatment groups).

### Example 3

### Intranasal Administration of Oxytocin as Treatment to Decrease Alcohol Craving and Relapse in Patients with a History of Alcohol Dependence or Abuse

### Subjects:

Have the same inclusion and exclusion criteria as subjects described in Example 2 and have just been discharged from the hospital after successful medical detoxification from alcohol using standard treatment. They do not receive standard medication treatments to decrease craving and relapse upon discharge (disulfuram, naltrexone, acamprosate).

### Study Design:

This is a double-blind, placebo controlled comparison of the efficacy of twice daily intranasal administration of oxytocin and placebo in prolonging sobriety, improving compliance with attending outpatient alcoholism therapy sessions and decreasing the amount of alcohol consumed after inpatient medical detoxification from alcohol. Each treatment group is composed of up to 40 subjects. Treatment group assignment is random within each sex and follows a randomization scheme devised by a biostatistician.

### Procedure:

Subjects are recruited and sign consent forms toward the end of their inpatient medical detoxification. They agree to individual outpatient therapy with an experienced substance abuse therapist to assist them in remaining sober. Therapy is weekly for 4 sessions and then every other week. Subjects are instructed in intranasal self-administration of test substances by inpatient nursing staff at the end of their hospitalization. They are discharged from the hospital with an intranasal spray vial containing enough test substance to last until their outpatient appointment. Prior to discharge, subjects self administer one dose of test substance and then self administer one dose in the morning (before or after breakfast) and in the early evening (before or after dinner). Each dose consists of 6 insufflations; each administered 30 seconds apart and alternating between nostrils. Those in the oxytocin treatment group receive 24 international units per dose.

At the beginning of each therapy session, the therapist elicits information from subjects using the Timeline Followback Instrument (TLFB, Sobell et al., Brit. J. Addict. 83:393-402 (1988)) to ascertain whether subjects have consumed alcohol since discharge or the last therapy session, on which days they drank and how much. Breathalyzer measures are obtained and urine collected at these sessions for measurement of illicit drugs. In addition, subjects rate their craving for alcohol by completing the Obsessive Compulsive Drinking Scale and/or the Penn Alcohol Craving Scale as well as indicating on 100 mm visual analog scales how much they “want,” “need,” and “crave” alcohol. At the end of each therapy session, each subject is given a new intranasal spray vial containing enough of their assigned test treatment to last them until their next therapy session.

### Example 4

### Intranasal Administration of Oxytocin as a Treatment for Opioid Withdrawal

### Subjects:

Patients admitted to an inpatient unit at UNC Hospitals for medical detoxification from opioid (also referred to as opiate) drugs employing abrupt cessation of opioids and administration of other drugs to control withdrawal symptoms.

### Inclusion Criteria:

1) Daily consumption of doses of opioid drugs equivalent to 20 mg of morphine or more for at least 2 weeks prior to the current admission. 2) At least one prior episode 2 days or longer in duration during which the subject experienced opioid withdrawal symptoms that caused significant incapacitation (e.g., unable to work, do other normal activities) OR at least one prior inpatient or outpatient medical detoxification during which the subject exhibited withdrawal symptoms of sufficient magnitude that medication was required to control symptoms at least once on 2 consecutive days after cessation of or reduction in the amount of opioid consumed following 2 weeks or more of opioid drugs equivalent to 20 mg of morphine or more for at least 2 weeks.

### Exclusion Criteria:

1) Current delirium, disorientation to place or person, seizures, psychosis, mania, dependence on substances other than opioids (abuse of other substances is not exclusionary), suicidal or homicidal ideation with strong intent, plans or recent attempt (ideation alone is not exclusionary); history of primary psychotic disorder (e.g., schizophrenia, schizoaffective disorder, delusional disorder); amnesia, dementia, cognitive impairment, significant neurological symptoms, compromising medical conditions (e.g., AIDS [HIV infection alone as well as adequately controlled conditions such as diabetes, hypertension, asthma are not exclusionary], seizure disorder, emphysema, cancer); low body weight (BMI<17); history of anorexia nervosa or bulimia in the past 2 years; significant trauma, self injurious behavior or surgery in the previous 2 months; pregnancy; breast-feeding, parturition or breast-feeding in the past 6 months; 2) ingestion during the 2 weeks prior to this admission of much more opioid/day than during previous periods of opioid consumption that preceded the onset of withdrawal symptoms, 3) the last dose of opioid drug was more than 18 hours prior to enrollment in the test treatment trial, 4) administration of drugs to control withdrawal symptoms for more than 12 hours prior to enrollment in the study, 5) vomiting and/or diarrhea that is sustained and copious enough to produce dehydration requiring intravenous administration of fluids, 6) vital signs prior to recruitment into the study are SBP greater than 190, DBP greater than 125, heart rate greater than 140 beats/minute or body temperature greater than 40 degrees Centigrade, 7) any of the following laboratory values on admission: AST>110 U/L (normal range 19-55), ALT>140 U/L (normal range 19-72), alkaline phosphatase >225 U/L (38-126), GGT>200 U/L (normal range 13-68), non-fasting glucose >250 mg/ml (normal range 65-179), hematocrit <38% (normal range 41-53), hemoglobin <12 g/dl (normal range 13.5-17.5) or any other laboratory value that is outside the normal range including electrolytes or EKG abnormalities (mild-moderate liver function test elevations or glucose levels in diabetics are not exclusionary), 8) inability to read well enough to complete study questionnaires (this is determined by whether the prospective subject can read and understand the consent form without help). Concurrent depressive and/or anxiety disorders without psychotic features are not exclusionary. Standing or PRN medications for non-exclusionary disorders are permitted.

### Study Design:

This is a double-blind, placebo-controlled comparison of the efficacy of twice daily intranasal administration of oxytocin and placebo in reducing the number and magnitude of opioid withdrawal symptoms, the number of times withdrawal symptoms increase in magnitude sufficiently to require medication administration to control those symptoms and the total amount of PRN medications given during the course of inpatient medical detoxification. Treatment group assignment is random within each sex. Each treatment group is composed of up to 40 subjects. Subjects are assigned to treatment groups according to a randomization scheme devised by a biostatistician.

### Procedures:

Upon admission to UNC Hospitals, all patients have their VSs measured and their withdrawal symptoms rated using the Subjective Opiate Withdrawal Scale (SOWS, Handelsman et al., Am J Drug Alcohol Abuse 13: 293-308 (1987)) at approximately 4 hour intervals (0600, 1000, 1400, 1800, and 2200 hours) or whenever patients report or nursing or medical staff notice intensification of withdrawal symptoms. On the SOWS, each of 16 opioid withdrawal symptoms are rated as 0=not at all, 1=a little, 2=moderately, 3=quite a bit, 4=extremely. Experienced nursing staff use patient report and their clinical observations to complete the SOWS ratings. In addition, patients who are admitted for detoxification are checked by nursing staff every 15 minutes around the clock for the first 2 days of their admission and then every 30 minutes on subsequent admission days. Except when patients are asleep at night, they are asked during each check if they are experiencing increased withdrawal symptoms.

Most patients who are admitted to the UNC Hospitals for medical detoxification come in through the Emergency Department during the late evening or early morning. Physical examination, medical and psychiatric history and lab tests (including CBC, electrolytes including magnesium, BUN, creatinine, glucose, albumin, ALT, AST, LDH, GGT, TSH, B12, folate, urinalysis, urine toxicology screen and pregnancy test) are routinely obtained during this admission process. As is standard procedure in managing patients admitted for opioid detoxification, lab values and physical examination results obtained the evening/early morning before as well as VSs and SOWS ratings obtained since admission are reviewed the morning after admission by the attending physician who also interviews the patient during rounds to confirm and obtain more detailed information about medical and psychiatric history and in particular past opioid withdrawal history and recent opioid drug intake. Subjects who meet inclusion but not exclusion criteria have this study briefly explained to them. If they express interest in participating, a member of the research team describes the study in more detail and, if the patient continues to express interest, obtain informed consent. An ECG is then performed.

During admission, the following doses of medication are given as often as every 4 hours to control the following opioid withdrawal symptoms when rated as 3 or greater on the SOWS: promethazine 25 mg for nausea and/or vomiting; loperamide 4 mg for diarrhea; dicyclomine 10 mg for abdominal cramps; cyclobenzaprine (Flexeril) 10 mg for muscle cramps/twitches; and acetaminophen 650 mg for muscle/bone pain; lorazepam 1 mg for anxiety; diphenhydramine 25 mg for rhinorrhea/lactimation. Each of these medication doses is repeated in 1 hour if the symptom rating continues to be 3 or greater. Clonidine 0.1 mg is also given if two or more symptoms are rated 3 or higher (systolic/diastolic blood pressures must be greater than 110/65 to give clonidine). Clonidine doses may be given every 6 hours if necessary.

After consent is obtained, subjects are randomly assigned to a test treatment group and promptly receive their first intranasal treatment. This usually occurs between 1000 and 1200 hours and consists of 6 insufflations of SYNTOCINON® Spray (approximately 24 IU of oxytocin) or placebo each insufflation given 30 seconds apart and alternating between nostrils. Test treatments are administered from intranasal spray vials designed to deliver 0.1 ml metered volume per insufflation. Vials containing oxytocin and placebo spray are relabeled by the UNC Investigational Drug Service so that subjects and raters are blind to treatment. We only recruit subjects admitted late the previous evening or early morning (this is by far the majority of patients admitted for medical detoxification). These subjects receive their first dose of intranasal test substance by mid-late morning.

After their initial test dose, subjects receive intranasal test treatments at 0900 and 1700 hours through the first 3 full days of inpatient admission. Subjects self-administer doses after instruction from and under the supervision of nursing staff. VSs, SOWS ratings continue to be obtained at approximately 0600, 1000, 1400, 1800, and 2200 hours or whenever patients report or clinical staff members observe evidence of intensification of withdrawal symptoms. On day 2, one hour after 0900 dose, another EKG is obtained. On subsequent inpatient days (admission day 4 onward), subjects no longer receive intranasal test treatments and VSs and SOWS ratings are obtained bid (1000 and 1800 hours) or whenever patients report intensification of withdrawal symptoms.

Subjects rate their craving for opioid drugs during the morning on the second and subsequent days of their inpatient detoxification on the modified Heroin Craving Questionnaire (Schuster et al., Exp Clin Psychopharmacol 3: 424-431 (1995); Heinz et al., J Subst Abuse Treat 31:355-364 (2006)) as well as indicating on 100 mm visual analog scales how much they “want,” “need,” and “crave” opioid drugs.

### Example 5

### Intranasal Administration of Oxytocin as a Treatment to Decrease Opioid Craving and Relapse

### Subjects:

Subjects have the same inclusion and exclusion criteria as subjects described in Example 4 and have just been discharged from the hospital after successful opioid medical detoxification using the standard medications described in Example 4 to control withdrawal symptoms. They do not receive SUBOXONE® (buprenorphine+naltrexone) which has come into widespread use as a treatment to decrease craving and relapse.

### Study Design:

This is a double-blind, placebo controlled comparison of the efficacy of twice daily intranasal administration of oxytocin and placebo in prolonging sobriety, improving compliance with attending outpatient opioid dependence therapy sessions and decreasing the amount of opioid drugs consumed after inpatient medical detoxification. Each treatment group is composed of up to 40 subjects. Treatment group assignment is random within each sex and follow a randomization scheme devised by a biostatistician.

### Procedure:

Subjects are recruited and sign consent forms toward the end of their inpatient medical detoxification. They agree to individual outpatient therapy with an experienced substance abuse therapist to assist them in remaining sober. Therapy is weekly for 4 sessions and then every other week. Subjects are instructed in intranasal self-administration of test substances by inpatient nursing staff at the end of their hospitalization. They are discharged from the hospital with an intranasal spray vial containing enough test substance to last until their outpatient appointment. Prior to discharge, subjects self administer one dose of test substance and then self administer one dose in the morning (before or after breakfast) and in the early evening (before or after dinner). Each dose consists of 6 insufflations; each administered 30 seconds apart and alternating between nostrils. Those in the oxytocin treatment group receives 24 international units per dose.

At the beginning of each therapy session, the therapist elicits information from subjects using the Timeline Followback Instrument (TLFB, Sobell et al., Brit. J. Addict. 83:393-402 (1988)) to ascertain whether subjects have taken opioid drugs since discharge or the last therapy session, on which days they used and how much. Urine is collected at these sessions for measurement of opioid as well as illicit drugs. In addition, subjects rate their craving for opioids by completing the modified Heroin Craving Questionnaire as well as indicating on 100 mm visual analog scales how much they “want,” “need,” and “crave” opioid drugs. At the end of each therapy session, each subject is given a new intranasal spray vial containing enough of their assigned test treatment to last them until their next therapy session.

### Example 6

### Oxytocin Treatment of Withdrawal, Craving and Relapse in Other Addictive Disorders

Diagnostic criteria for cocaine, benzodiazepine, nicotine and gambling dependence and withdrawal are summarized in the DSM-IV-R. Most patients who have these dependence disorders continue to experience craving and are at risk for relapse after undergoing medical detoxification or psychotherapy to control withdrawal symptoms after cessation of ingestion of these substances or curtailment of gambling. Subjects with these disorders are given oxytocin treatment (e.g., intranasally) to treat withdrawal symptoms, decrease craving and/or reduce relapse. For example, the effectiveness of twice daily intranasal administration of oxytocin (24 international units per dose) is compared with placebo for treating one of these disorders in a double-blind trial in which withdrawal, craving and relapse are quantified using well-established rating instruments.

### Example 7

### Oxytocin Treatment of Social Deficits and other Psychotic Symptoms in Schizophrenia: a Two-Week Trial

### Subjects:

Twenty-three subjects were initially enrolled; 2 dropped out before randomization to treatment and 1 dropped out early because he developed an upper respiratory infection; the remaining 20 completed the protocol and constitute our study sample. Inclusion criteria were: 18-55 years of age, DSM-IV diagnosis of paranoid or undifferentiated schizophrenia ≧1 year, PANSS total score ≧60, PANSS suspiciousness/persecutory item score ≧4 or 3 on this item and ≧3 on at least one other social behavior-relevant PANSS item (hostility, passive/apathetic social withdrawal, uncooperativeness, active social withdrawal), treatment with one or more standard antipsychotic medications, stability of medication doses and symptoms ≧1 month.

The study was approved by the University of North Carolina Biomedical Institutional Review Board and conducted in accordance with The Code of Ethics of the World Medical Association. Written informed consent was obtained from all subjects.

### Procedures

This was a randomized, double blind, placebo-controlled 2-week treatment trial. Screening of each subject included a review of psychiatric and medical history, physical examination, an ECG, and blood and urine collection for standard laboratory tests. Within 1 week after screening, baseline social cognition measures were obtained followed by psychiatric ratings. Daily intranasal treatments were initiated after baseline assessments. Social cognition measures and psychiatric ratings were repeated beginning 50 minutes after the AM dose of study medication on treatment day 14. The social cognition instruments were the Brüne Theory of Mind Picture Stories Task (Brüne, (2003) Social cognition and behaviour in schizophrenia. In Brüne, Ribbert, Schiefenhövel (eds.) The social brain-evolution and pathology. John Wiley & Sons; Chichester, pp. 277-313) and the Trustworthiness Task (Adolphs et al., Nature 393: 470-474 (1998)). Psychiatric measures included the Positive and Negative Symptom Scale (PANSS) (Kay et al., Schizophr. Bull. 13: 261-276 (1987))) and the Paranoia Scale (Fenigstein & Vanable, J. Pers. Soc. Psych. 62: 129-138 (1992))). The first 10 subjects were studied as inpatients on a clinical research unit and the last 10 subjects were studied in an outpatient research clinic. Blood and urine laboratory tests, ECGs and body weights were obtained at screening and treatment day 14 in all subjects and also on treatment days 3 and 7 in inpatients.

### Study Drugs

Subjects remained on their pre-study medication regimen and doses throughout the treatment trial. They self-administered intranasal study drug twice daily; before breakfast and before dinner. Each dose consisted of six 0.1 ml insufflations (alternating between the left and right nostril) of oxytocin spray containing approximately 24 international units of oxytocin (SYNTOCINON® Spray, Novartis) or placebo. Outpatient compliance with test treatments was monitored by weighing spray vials before they were dispensed and after the morning dose on treatment day 14.

### Statistical Analyses

All dependent variables were examined for normality and skewness prior to analyses. We compared oxytocin and placebo group means using an unpaired t test for the following baseline variables: demographic, psychiatric history and diagnosis, inpatient versus outpatient status, social cognition and psychiatric measures. We performed a paired t test within each treatment group examining changes from baseline to two weeks on the following variables: biological (laboratory test values, vital signs), social cognition measures (Brüne Task, Trustworthiness Task), and psychiatric measures (PANSS total and subscales, Paranoia Scale). To compare the treatment groups over time on social cognition and psychiatric variables, we used analysis of covariance controlling for the baseline measure of the dependent variable.

### Results

The sample consisted of 17 men and 3 women: 10 Caucasian and 10 African-American. There were no significant demographic, social cognition or psychiatric history differences between the treatment groups (Table 2). Neither treatment group had clinically or statistically significant changes over the 14-day treatment period in laboratory safety measures (CBC, electrolytes, glucose, BUN, creatinine, liver functions, urinalysis), ECGs or vital signs.

Table 3 summarizes the results. There were no differences between treatment groups at baseline on any of the outcome variables. The oxytocin group had significant improvements from baseline to treatment day 14 in accurate identification of second order false belief in the Brüne Task as well as significant reductions in PANSS total, positive subscale, general subscale, suspiciousness/persecutory item, anxiety item and Paranoia Scale scores. In addition, oxytocin recipients showed trends toward significant improvement in accurate recognition of deception in the Brüne Task and rating untrustworthy faces (faces rated by a normative sample as untrustworthy) as less untrustworthy. The oxytocin group had no changes that approached significance in other Brüne Task measures that have been reported to differ between patients with schizophrenia and healthy controls (second order belief, third order false belief, recognition of cheating) (Brüne, Psychiatry Res. 133:135-147 (2005)). In the placebo group, the only significant change during the treatment period was a decline in PANSS suspiciousness item scores.

TABLE 2
Descriptive Data for Oxytocin (OT) and Placebo Participants

| | | |
|---|---|---|
| | OT | Placebo |
| Variable | (n = 11) | (n = 9) |

| | N | % | N | % |
|---|---|---|---|---|
| Male | 9 | 82 | 8 | 89 |
| Ethnicity | | | | |
| African- | 5 | 45 | 5 | 56 |
| American | | | | |
| Caucasian | 6 | 55 | 4 | 44 |
| Diagnosis | | | | |
| PS | 10 | 91 | 7 | 78 |
| US | 1 | 9 | 2 | 22 |
| Inpatient | 5 | 45 | 5 | 56 |
| | Mean | SD | Mean | SD |
| Age | 39.00 | 11.18 | 35.78 | 9.52 |
| Years of Education | 12.27 | 1.01 | 12.56 | 1.51 |
| Years of Illness | 16.00 | 11.08 | 12.89 | 9.43 |
| Hospitalizations | 7.82 | 7.90 | 7.11 | 7.93 |

PS = paranoid schizophrenia; US = undifferentiated schizophrenia; Years of Illness = Number of years since diagnosis of schizophrenia; Hospitalizations = Number of psychiatric hospitalizations
OT = oxytocin treatment; AA = African American; CA = Caucasian; PS = paranoid schizophrenia; US = undifferentiated schizophrenia; Inpt = inpatient when studied; Outpt = outpatient when studied

ANCOVAs controlling for baseline revealed that the oxytocin group (Least squared [LS] mean=70.56; SE=2.07), compared to the placebo group (LS mean=77.21; SE=2.29), had a significantly greater decline in PANSS total scores (F=4.60, p=0.047). There was also a trend toward a significantly greater decline in PANSS general subscale scores (F=3.78, p=0.069) for the oxytocin group (LS mean=34.34; SE=1.35) compared to placebo (LS mean=38.26; SE=1.49). There were no group differences in change over the treatment period for other PANSS or social cognition variables.

TABLE 3
Outcome Measures: Means, Standard Deviations and Ranges

| | Oxytocin | | | | Placebo | | | |
|---|---|---|---|---|---|---|---|---|
| | (n = 11) | | | | (n = 9) | | | |
| | Baseline | | Day 14 | | Baseline | | Day 14 | |
| | M (SD) | Range | M (SD) | Range | M (SD) | Range | M (SD) | Range |
| Brüne false belief | 1.91 (.83) | 1-3 | 2.45 (.69)^{c} | 1-3 | 2.11 (.60) | 1-3 | 2.33 (.87) | 1-3 |
| Brüne deception | 2.27 (.79) | 1-3 | 2.82 (.40)^{a} | 2-3 | 2.22 (.83) | 1-3 | 2.44 (.73) | 1-3 |
| Trustworthiness | −15.82 (15.03) | −36-+20 | −9.36 (12.33)^{a} | −36-+11 | −3.33 (12.19) | −18-+15 | −6.44 (12.47) | −19-+17 |
| PANSS total score | 82.91 (13.59) | 61-101 | 71.82 (11.29)^{d} | 51-86 | 78.89 (16.75) | 58-110 | 75.67 (13.73) | 57-106 |
| PANSS positive | 20.46 (5.80) | 9-30 | 17.73 (5.44)^{c} | 9-26 | 21.67 (6.91) | 13-30 | 20.44 (5.50) | 13-27 |
| PANSS negative | 21.00 (4.12) | 13-28 | 18.91 (3.81)^{a} | 13-28 | 19.11 (3.98) | 13-26 | 18.00 (3.67) | 12-24 |
| PANSS general | 40.64 (7.84) | 28-49 | 35.18 (6.48)^{c} | 24-44 | 38.22 (10.41) | 25-58 | 37.22 (10.08) | 24-59 |
| PANSS suspicious | 4.45 (0.69) | 3-5 | 3.73 (1.01)^{c} | 2-5 | 4.22 (.97) | 3-6 | 3.78 (.83)^{b} | 3-5 |
| PANSS anxiety | 2.73 (1.19) | 1-5 | 1.91 (1.58)^{b} | 1-5 | 3.44 (1.67) | 1-6 | 2.89 (1.54) | 1-6 |
| Paranoia Scale | 48.73 (21.53) | 23-82 | 44.91 (21.48)^{b} | 22-82 | 42.56 (14.83) | 20-63 | 39.44 (16.75) | 20-66 |

Range = Observed range of scores; Brüne = Brüne Theory of Mind Picture Stories Task; Brüne false belief = 2nd order false belief identification score
on Brüne Task; Brüne deception = deception identification score on Brüne Task; Trustworthiness = Trustworthiness Task score on untrustworthy
faces (scored below the mean by normative group); PANSS = Positive and Negative Symptom Scale; PANSS positive = positive symptom subscale
score on PANSS; PANSS negative = negative symptom subscale score on PANSS; PANSS general = general symptom subscale score on PANSS;
PANSS suspicious = PANSS suspiciousness/persecutory item score; PANSS anxiety = PANSS anxiety item score.
Superscripts indicate significant within-group differences using paired comparisons t-tests between baseline and Day 14.
a = p ≦.08, b = p <.05, c = p <.01, d = p <.001; Day 14 compared to Baseline.

These results are the first indicating that oxytocin treatment reduces social cognition deficits in schizophrenia. The improvements in social cognition in oxytocin recipients are particularly exciting. Deficits in social cognition have been strongly linked to social dysfunction (Couture et al., Schizophr. Bull. 32: S44-SS63 (2006); Fett et al., Neurosci. Biobehav, Rev. 35:573-588 (2011)), the major cause of disability in schizophrenia and the aspect of the disorder that is least responsive to currently available antipsychotic medications (Penn et al., Schizophr Res 115(1): 17-23 (2009)). Therefore, improvements in social cognition associated with oxytocin treatment could potentially be accompanied by improvements in social functioning. dysfunction, the major cause of disability in schizophrenia (Penn et al, Schizophr. Bull. 34:408-411 (2008); Phillips et al., Biol. Psychiatry. 54:515-528 (2003); Yager et al., Psychiatry 69: 47-68 (2006)). Three large clinical trials (including the CATIE trial) as well as other studies found that typical and atypical antipsychotic medications did not improve social cognition or social functioning (Bellack et al., Am. J. Psychiatry 161: 364-367 (2004); Buchanan et al., Am. J. Psychiatry 155:751-760 (1998); Kirkpatrick et al., Neuropsychopharmacology 22:303-310 (2000); Penn et al., Schizophr. Res, 115: 17-23 (2009); Harvey et al., Am. J. Psychiatry 163: 1918-25 (2006); Sergi et al., Am. J. Psychiatry 164: 1585-1592 (2007)). Oxytocin may prove to be uniquely effective in reducing social dysfunction in schizophrenia by improving social cognition.

### Example 8

### Expanded Study of Two Weeks of Intranasal Oxytocin Treatment of Schizophrenia

We have expanded our double blind, placebo-controlled pilot study described in Example 7 to include 25 subjects with schizophrenia in which we compared the effects of 14 days of twice daily intranasal oxytocin (24 IU/dose, N=14) vs. placebo (N=11) administration on PANSS (Positive and Negative Symptom Scale) total and PANSS item scores, subject self-ratings on the Paranoia Scale and performance on social cognition tests (the Brüne Theory of Mind Picture Stories Task and the Trustworthiness Task). As in the smaller study, all subjects' symptoms and psychotropic medication regimens were stable for ≧1 month prior to the treatment trial. Medication doses remained the same during the study period.

There were no significant demographic or psychiatric history differences between the treatment groups (age, gender, race, years of education, years since onset of illness, diagnosis [chronic vs. undifferentiated schizophrenia], number of hospitalizations). Neither treatment group had significant changes over the 14 day treatment in laboratory tests (CBC, electrolytes, glucose, BUN, creatinine, liver functions, UA), EKGs, vital signs.

Table 4 below summarizes the means and standard deviations at baseline and treatment day 14 for psychotic symptom measures (PANSS total, positive subscale, negative subscale, positive subscale scores as well as Paranoia Scale scores). as well as the with-in group t-test p values for changes in the measures in the oxytocin and Placebo groups separately. In the oxytocin group, all psychotic measures decreased significantly over the 2-week treatment trial. In the Placebo group, there was only a trend toward a significant decline in Paranoia Scale scores.

TABLE 4
Psychotic Symptoms: Oxytocin (N = 14) and Placebo (N = 11)
Within Group Results

| | Baseline | | Tx Day 14 | | | |
|---|---|---|---|---|---|---|
| | M | SD | M | SD | t | p |
| OXYTOCIN | | | | | | |
| PANSS | 79.00 | 14.23 | 69.21 | 11.78 | 4.30 | .0009 |
| total score | | | | | | |
| PANSS positive | 19.79 | 5.26 | 17.14 | 5.11 | 3.62 | .003 |
| score | | | | | | |
| PANSS negative | 20.36 | 4.20 | 18.00 | 3.84 | 2.54 | .025 |
| score | | | | | | |
| PANSS general | 38.21 | 8.55 | 34.07 | 6.72 | 2.95 | .011 |
| score | | | | | | |
| Paranoia Scale | 48.00 | 20.15 | 45.00 | 20.28 | 2.41 | .032 |
| PLACEBO | | | | | | |
| PANSS total | 78.00 | 15.14 | 74.36 | 12.71 | 1.55 | .152 |
| score | | | | | | |
| PANSS positive | 21.36 | 6.22 | 20.18 | 4.98 | 1.36 | .205 |
| score | | | | | | |
| PANSS negative | 18.27 | 4.03 | 17.27 | 3.69 | 1.12 | .290 |
| score | | | | | | |
| PANSS general | 38.45 | 9.35 | 36.91 | 9.07 | 1.04 | .325 |
| score | | | | | | |
| Paranoia Scale | 47.00 | 17.03 | 43.09 | 19.14 | 1.85 | .095 |

Table 5 below summarizes the means and standard deviations at baseline and treatment day 14 for social cognition measures (Brüne 2^{nd} order false belief, Brüne 3^{rd} order false belief, Brüne deception recognition, Trustworthiness task) as well as the with-in group t-test p values for changes in the measures in the oxytocin and Placebo groups separately. In the oxytocin group, all of these measures declined significantly or nearly significantly over the 2-week treatment period. In the Placebo group, none of these measures changed significantly.

TABLE 5
Social Measures: Cognition and PANSS

| | |
|---|---|
| Baseline | Tx Day 14 |

| | M | SD | M | SD | t | p |
|---|---|---|---|---|---|---|
| OXYTOCIN | | | | | | |
| Brüne 2^{nd} false belief | 1.93 | .83 | 2.29 | .83 | −2.11 | .055 |
| Brüne 3^{nd} false belief | 1.93 | 1.07 | 2.43 | .94 | −2.19 | .048 |
| Brüne deception | 2.43 | .76 | 2.86 | .36 | −1.88 | .082 |
| Trustworthiness task* | −15.71 | 13.90 | −10.36 | 11.61 | −1.96 | .072 |
| PANSS social items | 2.91 | .62 | 2.39 | .53 | 5.08 | .0002 |
| mean⁺ | | | | | | |
| PLACEBO | | | | | | |
| Brüne 2^{nd} false belief | 2.27 | .65 | 2.45 | .82 | −1.00 | .341 |
| Brüne 3^{nd} false belief | 2.55 | .52 | 2.55 | .82 | 0.00 | 1.000 |
| Brüne deception | 2.36 | .81 | 2.55 | .69 | −1.00 | .341 |
| Trustworthiness task* | −6.18 | 12.68 | −7.27 | 11.38 | 0.30 | .769 |
| PANSS social items | 2.75 | .82 | 2.53 | .78 | 1.71 | .119 |
| mean⁺ | | | | | | |

* = ratings of untrustworthy faces (below the mean in normal sample)
+ = suspiciousness, hostility, social withdrawal (passive, active), uncooperativeness

Because of our interest in oxytocin effects on social function in schizophrenia, we focused our statistical analysis on an a priori subset of PANSS items: P6 (suspiciousness/persecution [paranoia]), P7 (hostility), N4 (passive/apathetic social withdrawal), G8 (uncooperativeness) and G16 (active social avoidance). These items were used as inclusion criteria for this study (see description in Example 7). Values on these items are highly correlated in our sample (Cronbach's alpha=0.84). As summarized in Table 5, the mean PANSS social item scores dropped very significantly over the 2-week treatment trial in the oxytocin treatment group but did not change significantly in the Placebo group.

ANOVAs revealed that the oxytocin group had a significantly greater decline in PANSS total score than the Placebo group during the treatment trial (F=4.29, p=0.05). Also, the oxytocin group compared to the Placebo group had trends toward significantly greater declines in PANSS social items mean scores (F=3.15, p=0.0898) and in PANSS positive symptom subscale scores (F=3.10, p=0.0923).

### Example 9

### Oxytocin Treatment of Social Cognitive and Functional

### Deficits in Schizophrenia (12 Week Treatment Trial)

### Subjects:

54 subjects, 18-55 years of age, who have had DSM-IV schizophrenia for at least one year. There is no selection on the basis of gender, ethnicity or race.

### Inclusion/Exclusion Criteria

### Inclusion Criteria:

18-55 yrs of age; currently meeting DSM-IV criteria for schizophrenia with onset occurring >1 year prior to enrollment in the study; PANSS total score >60; score of <22 on the Reading the Mind in the Eyes Test (“Eyes Test”, Baron-Cohen et al., J Child Psychol. Psychiatry 42: 241-251 (2001)), which represents 1 SD below the mean in a large normative sample; and stable symptoms as well as being on the same medication and psychosocial therapy regimen for >1 month. The Eyes test maximum score may be changed to 0.5 SD below the normal mean depending on the impact on recruitment success of using the score of <22.

Several considerations lead to the use of the Eyes Test score and the total PANSS score as inclusion criteria. The Eyes Test is a measure of both theory of mind and emotion recognition (see description of this test below), which are two major areas of social cognition deficiency in schizophrenia. Using as an inclusion criterion a maximum score on the Eyes Test of 1 SD (or 0.5 SD) below the mean in healthy individuals ensures that subjects have significant social cognition deficits that allows a more robust test of the primary hypothesis that OT treatment improves social cognition. Requiring a PANSS total score >60 also assures that subjects have sufficient psychotic symptoms that the secondary hypothesis that OT treatment significantly decreases those symptoms can adequately be tested.

### Exclusion Criteria:

Current or lifetime history of schizoaffective, delusional, other psychotic (shared, substance-induced, due to a medical disorder) disorders, bipolar, cyclothymic, somatoform, dissociative, eating or personality disorders, unipolar major depressive episodes with psychotic features, dementia; substance use or abuse disorder during the past 3 months (except tobacco, caffeine); treatment currently or within the past 6 months with high dose sedative-hypnotics, stimulants, chronic glucocorticoids (other medications that are adequately controlling acute or chronic disorders [e.g., hypertension, diabetes, hypo or hyperthyroidism, asthma, allergies, mild infections, etc.] are allowed); debilitating or inadequately controlled medical conditions (HIV infection without AIDS is not exclusionary); major surgery/trauma in the past 4 months; pregnancy, childbirth or breast-feeding in the past year; significant physical exam, laboratory or EKG abnormalities; reading level <5th grade on the Wide Range Achievement Test (WRAT, Wilkinson (1993) The Wide Range Achievement Test 3 administrative Manual. Wilmington, Del.: Range, Inc.), inability to read well enough to complete study questionnaires (this will be determined by whether the prospective subject can read and understand the consent form without help)

### Study Design, Methods and Procedures

### Overview of Procedures:

All procedures are conducted at the UNC CTRC outpatient clinic or at the outpatient North Carolina Psychiatric Research Center (NCPRC), a specialized program of the University of North Carolina Center for Excellence in Community Mental Health and a unit within Central Regional Hospital in Raleigh, N.C. Except for Screening, all clinic visits begin between 0800-1000 hours with subjects having fasted since midnight. Approximately 10 minutes after checking into the clinic, subjects' weight and vital signs are measured followed by blood and urine sample collection. Breakfast is then be served. At regular intervals during each clinic visit, subjects are allowed to rest for 15-20 minutes. Lunch is also provided for the subjects.

### Screening Assessment:

During the initial clinic visit and after giving informed consent, prospective subjects' psychiatric and medical histories are reviewed, physical exams conducted, EKGs run and blood and urine collected for complete blood count (CBC), electrolytes, liver/renal function tests, TSH, glucose, urinalysis (UA), pregnancy test, and urine drug screen (UDS). The Structured Clinical Interview for DSM-IV is administered to confirm diagnoses. The WRAT is administered to determine reading ability as well as the Reading the Mind in the Eyes Test (Eyes Test) to determine if subjects meet the social cognition criterion for impairment. Finally, PANSS and Calgary Depression Scale ratings are obtained.

### Baseline and Treatment Week 6 and 12 Assessments:

All of the primary and secondary outcome measures are obtained during clinic visits at Baseline (occurring within 1 week after the Screening assessment) and at the end of the treatment trial (12-week time point). Some of the outcome measures are obtained at a clinic visit at the 6-week time point during the treatment trial. Subjects arrive at the clinic between 8:00 and 10:00 a.m. for each of these visits and are given breakfast. At Baseline, administration of assessments begin 10 min after completion of breakfast. At the 6 and 12-week clinic visits, assessments begin 50 min after intranasal administration of test treatment (which is given before breakfast-see details below). The primary outcomes are social cognition measures (details below): emotion recognition (ER-40, Eyes Test), theory of mind (Eyes Test, Brüne test), social perception (Trustworthiness Task) and attributional style (AIHQ). The secondary outcomes (details below) are social functioning (SLOF), social skill (role plays), psychotic symptoms (PANSS), paranoia (Paranoia Scale), empathy (IRI), non-social cognition (BACS), motivation (IMI), anxiety (BSI anxiety items, Liebowitz social anxiety scale) and depression (Calgary Depression Rating Scale).

While we ultimately want to improve social functioning, we acknowledge that this domain might be slower to improve than social cognition, thus rendering it a secondary rather than primary outcome (and for that reason, we are assessing social functioning only at baseline and week 12 (end of treatment)). We included non-social cognition in the battery to assess whether treatment effects on social cognition generalize to other domains of cognition. A similar sentiment underlies our inclusion of symptoms (both psychotic and general) in the battery, as the value of oxytocin may be enhanced if it can impact symptoms that might ultimately interfere with treatment engagement. Finally, we included empathy and motivation because of the relationship of the former with theory of mind, and the latter being a nonspecific factor that may influence performance on social cognition and social skills tests.

### Test Substance Administration and Other Procedures During the Treatment Trial:

During the treatment trial, subjects self-administer test treatments twice daily; before breakfast and before dinner. Each treatment consists of 6 insufflations (each 0.1 ml) of SYNTOCINON® Spray (Novartis), which contains 24 international units of oxytocin, or placebo solution containing all of the ingredients in SYNTOCINON® Spray except oxytocin. Blind-labeled test treatment vials are dispensed following a randomization scheme permuted in blocks of 4. Subjects self administer test treatments from 60 ml spray vials (ejecting 0.1 ml per spray) initially containing 35 ml of test substance. Each vial contains enough solution for 3 weeks of self administration. Subjects receive their first vial of test treatment right after assessments are complete at the Baseline clinic visit. They are instructed in intranasal self administration and care of test vials at that time and given written instructions on these matters. They take their first intranasal test dose under the observation of research nurses before leaving the clinic. Before leaving the clinic, subjects are given a card with the date of their return appointment in one week.

Subjects return to the clinic at the 1, 3, 6, 9 and 12 week time points during the treatment period. At the 1, 3, and 9-week visits, subjects have a brief visit with a study clinician who assesses side effects and performs a mental status examination. At the baseline, 6 and 12-wk time points, subjects arrive at the clinic between 8:00 and 10:00 a.m., having fasted overnight. Before eating breakfast, weight and vital sign measurements are obtained along with blood and urine samples. Blood collected at baseline is used to measure serum glucose and lipids and urine is used to screen for pregnancy in women and for drug screening in all subjects. Blood and urine samples obtained at the 6 and 12-week time points are used for the same measures obtained at the Screening visit. EKG is also repeated at the 6 and 12-week time points. Spot urine pregnancy tests are also done at 3 and 9 weeks. Prior to breakfast at the 6 and 12-wk time point visits, subjects self-administer their morning dose while observed by a research nurse to be sure the process is being done correctly. To obtain plasma for later assay of oxytocin concentrations, blood is drawn 40 minutes after the morning test dose into a chilled EDTA vacutainer tube which is immediately placed in an ice bath and spun down within 20 minutes. Plasma samples are stored at ultralow temperatures. oxytocin levels may reflect individual variability in oxytocin absorption and metabolism and are a covariate in statistical analyses.

Beginning 50 minutes after the morning test dose (6 and 12-week treatment time points), primary and secondary measures are re-administered.

Subjects bring their used test treatment vial to the clinic visits at the 3, 6, 9 and 12-week time points during the treatment trial. At the 3, 6 and 9 wk time points, subjects are given a fresh test treatment intranasal spray vial from which they continue twice daily self administration for the next 3 weeks.

Compensation for subjects after completing assessments is $20 at screening, $40 at baseline, $60 at the 6-week visit, $15 at the 1, 3, and 9-week visits, and $70 at treatment week 12 (total compensation=$235). Transportation costs are reimbursed for those who drive to and from the study sites at the rate of $0.45/mile. Cab fare is provided for each round trip for those who cannot or prefer not to drive or cannot find a ride. Parking costs are also reimbursed.

### Randomization Plan:

The statistician generates a randomization plan for randomizing 27 subjects to OT treatment and 27 subjects to placebo treatment using PROC PLAN in SAS Version 9.2. A blocksize of 4 is used within each sex so that every 4 subjects randomization to the two groups is equalized. The statistician then exports the randomization plan to a spreadsheet, which the data manager imports as a table into the study data management system.

When a subject is to be randomized, an authorized investigator, who does not need to be blinded, obtains the randomization number from the data management system. This authorized investigator then contacts the investigational drug service to give them the randomization number, and collects the appropriate medication from the investigational drug service.

### Monitoring and Enhancing Compliance:

Change in 60 ml spray vial weight over each 3 weeks of test treatment is used to monitor subject compliance. Each insufflation (0.1 ml) should decrease the vial weight by 0.1 gram. Subjects are called each weekday morning to remind them to take their intranasal test treatment. Less than 75% of the anticipated decline in vial weight may be grounds for termination from the study, especially if it occurs more than once.

### Monitoring Safety:

Laboratory tests, EKGs and vital signs are obtained at the 6 and 12-week time points. Abnormalities or adverse events may result in exclusion from further participation.

### Medical Evaluation

Screening:

During the initial clinic visit and after giving informed consent, prospective subjects' psychiatric and medical histories are reviewed, physical exams conducted, EKGs run and blood and urine collected for CBC, electrolytes, liver/renal function tests, TSH, glucose, UA, pregnancy test, and drug screen.

### At Baseline and During the Treatment Trial:

An extensive set of lab values, EKGs and vital signs are obtained during clinic visits at 6 and 12 weeks during the test treatment period (serum glucose and lipid measures will also be obtained at baseline). Subjects have been fasting since midnight and have not yet taken their morning test dose when their blood is drawn and urine collected shortly after they arrive for the baseline, week-6 and week-12 clinic visits. Running laboratory tests on blood and urine samples obtained in the fasting state, at these time points, is optimal for rapid detection of negative effects of oxytocin treatment on subjects' physiology. It may also provide preliminary information about the influence of oxytocin on the adverse metabolic effects of the atypical antipsychotic medications that most subjects are taking throughout the study period. Vital signs re repeated 2 hours after subjects take their morning intranasal test dose at the 6 and 12-week time points. Spot urine pregnancy tests are obtained at screening, baseline, and weeks 3, 6, 9 and 12 clinic visits during the treatment trial.

### Social Cognition Measures (Primary Outcomes)

The Emotion Recognition-40 Task (ER-40, Kohler et al., CNS Spectr. 9: 267-274 (2004)) consists of 40 faces presented sequentially on a computer screen along with the choices of rating the face as happy, sad, anger, fear or no emotion. It uses racially and ethnically diverse face images and is a psychometrically sound measure of social cognition in this clinical population (Carter et al., Schizophr. Bull. 35: 153-162 (2009)).

The Brüne Theory of Mind Stories Task (Brüne, (2003) Social cognition and behaviour in schizophrenia. In Brüne, Ribbert, Schiefenhövel (eds.) The social brain-evolution and pathology. John Wiley & Sons; Chichester, pp. 277-313) involves a series of 6 sets of 4 cartoon pictures that illustrate interactions between two or more individuals. The subject is asked to rearrange the pictures, initially presented in an illogical sequence, in an order that conveys a logical story. The period of time the subject takes to complete the task and the accuracy of the sequencing is recorded. Then, after the subject correctly organizes the pictures (which is done by the examiner if the subject's response is incorrect), the subject is asked questions about the cartoon characters' own beliefs and beliefs of other characters in the cartoons. The subject's interpretations of the characters' beliefs are scored as correct or incorrect.

The Reading the Mind in the Eyes test (Eyes Test, Baron-Cohen et al., J. Child Psychol. Psychiatry 42: 241-251 (2001)) consists of 36 photographs and participants are asked to guess the mental state (i.e., what the person is thinking or feeling) from among 4 choice words. Participants are given a practice item to ensure that they understand the task. Each eye region is presented on a note card or is displayed on a computer screen with the four choice mental states shown in the four corners of the card or computer screen (one target word and three foil words). There is no time constraint in choosing the mental state. A glossary of the mental states is made available if the participants are unsure of the meaning of a word. Performance is measured by the number of faces correctly discriminated.

The Trustworthiness Task (Adolphs et al., Nature 393: 470-474 (1998)) is comprised of 42 faces of unfamiliar people. Participants will be shown each picture individually (on a computer monitor) and will be asked to rate how much they would trust that person (i.e., with their money or their life) on a 7-point scale, ranging from −3 (very untrustworthy) to +3 (very trustworthy).

The Ambiguous Intentions Hostility Questionnaire (AIHQ, Combs et al., Cog. Neuropsychiatry 12: 128-143 (2007)) is comprised of 15 short vignettes that reflect negative events that vary in intentionality (i.e., obvious, accidental, and ambiguous intentions). Participants are asked to read each vignette, to imagine the scenario happening to her/him (e.g., “You walk past a bunch of teenagers at a mall and you hear them start to laugh”), and to write down the reason why the other person (or persons) acted that way toward her/him (as a means of measuring attributions). Two independent raters subsequently code this written response for the purpose of computing a “hostility bias”. The participant then rates, on Likert scales, whether the other person (or persons) performed the action on purpose (anchored by [1], definitely no, and [6], definitely yes), how angry it would make her/him feel (anchored by [1], not at all angry, and [5], very angry), and how much they would blame the other person (or persons) (anchored by [1], not at all, and [5], very much). Finally, the participant is asked to write down how she/he would respond to the situation, which is later coded by 2 independent raters to compute an “aggression index.”

### Social Function Measures (Secondary Outcomes)

The Specific Levels of Functioning Scale (SLOF, Schneider & Struening, Soc. Work. Res. Abstr. 19: 9-21 (1983)) is a 30-item questionnaire that has recently been found to be an excellent measure of social and general real-world functioning (Leifker et al., Schizoph. Res. 119: 246-52 (2010)). The questionnaire has 2 social functioning subsections (Interpersonal Relationships, Social Acceptability) and 2 community living skills subsections (Activities, Work Skills). One version is completed by the subject and another is completed by an informant, who will meet the following criteria: a) does not have a psychotic disorder, b) is literate, c) has known the subject for at least 1 year, and d) spends time with the subject on a regular basis (e.g., case worker, clinician, family member, caretaker). After giving informed consent, these criteria will be reviewed with each prospective informant. Informants will be compensated at $20/completed questionnaire. The informant questionnaire asks how well she/he knows the affected participant. Each item is rated on a 5-point Likert scale with anchors describing the frequency of the behavior and/or the patient's level of independence. To informants understand each item on the SLOF, this instrument will be administered by a member of the research team to the informant either in person or, if more convenient, over the phone.

The Social Competence test consists of two 90-second role-plays. The first role-play is an unstructured conversation in which the research confederate plays the role of a new neighbor with whom the subject is instructed to strike up a conversation. In the second role-play, the research confederate plays an upset friend who the subject is instructed to attempt to console. The role plays are recorded and later scored for conversational skills (e.g., overall social skills; affect; speech content, etc.) and ability to perceive distress in others (e.g., emotional empathy; cognitive empathy).

### Empathy and Motivation Measures (Secondary Outcomes)

The Interpersonal Reactivity Index of empathy (IRI, Davis, J. Personal Soc. Psychol. 44: 113-126 (1983)) is a self-report measure of cognitive and affective empathy. The IRI consists of 28 items where participants rate how well each item describes them using a five point scale. The 28 items yield four subscales: perspective taking (PT), empathic concern (EC), fantasy (F), and personal distress (PD). The PT subscale measures the tendency to take another's point of view (e.g., “I sometimes try to understand my friends better by imagining how things look from their perspective.”). The EC subscale measures feelings of sympathy and concern for others (e.g., “I often have tender, concerned feelings for people less fortunate than me.”). The F subscale measures the ability to imagine oneself in the role of a fictitious character in books (e.g., “When I am reading an interesting story or novel, I imagine how I would feel if the events in the story were happening to me.”). The PD subscale measures personal feelings of anxiety and unease in interpersonal settings (e.g., “Being in a tense emotional situation scares me.”). The IRI has been used previously to assess self-reported empathy in individuals with schizophrenia (Montag et al., Schizophr. Res. 92: 85-89 (2007)).

The Intrinsic Motivation Inventory (IMI) is a self report measure of intrinsic motivation. The original IMI (Ryan, J. Personal Soc. Psychol. 43: 450-461 (1982)) has recently been adapted for individuals with schizophrenia (Choi et al., Intrinsic Motivation Inventory: An adapted measure for schizophrenia research. Schizophr. Bull. Epub (2009)). The adapted IMI consists of 21 items rated on a 7 point Likert scale. The IMI taps into interest/enjoyment, value/usefulness and perception of choice (e.g., “I enjoy doing this activity very much”) in performing everyday tasks and activities, such as school, sports, medical procedures. Higher scores indicate more intrinsic motivation.

### Non-Social Cognitive Measures (Secondary Outcome)

The Brief Assessment of Cognition in Schizophrenia (BACS, Keefe et al., Schizophr. Res. 68: 283-297 (2004)) is an instrument that assesses verbal memory, working memory, motor speed, attention, executive functions and verbal fluency. To test verbal memory, subjects are asked to recall a list of 15 words five times. To test working memory, they are verbally presented a cluster of numbers of increasing length that they must repeat back to the rater in lowest to highest order. For motor function testing, subjects are given 100 plastic tokens and it is determined how many they can place in a container using both hands in one minute. They are also asked to use a symbol coding key to link within 90 seconds as many unique symbols as they can to specific numbers. Subjects' verbal fluency is tested by asking them to name in 60 seconds as many words as possible in a given category or with a particular starting letter. Executive functioning is tested by having the subject compare two pictures and state the minimum number of moves that would be required to rearrange the items in one picture to match the arrangement in the other picture. We selected the BACS over the MATRICS due to the study's emphasis on social cognition and the desire to reduce subject testing burden.

### Psychiatric Measures (Secondary Outcomes)

The Positive and Negative Symptoms Scale (PANSS, Kay et al., Schizophr. Bull. 13: 261-276 (1987)) is a 30-item scale on which an interviewer rates the subject for severity of positive and negative psychotic symptoms, and general and symptoms after asking a standard series of questions. Items are rated on a scale of 1 (absent) to 7 (severe), and yield five scaled scores: positive symptoms, negative symptoms, dysphoric mood, activation, and autistic preoccupation.

The Calgary Depression Rating Scale is an interviewer-rated measure of depression for individuals with schizophrenia (Addington et al., Br. J. Psychiatry 163(Suppl 22): 39-44 (1993)).

The Paranoia Scale (Fenigstein & Vanable, J. Pers. Soc. Psych. 62: 129-138 (1992)) is a questionnaire filled out by the subject on which he/she rates on a 1-5 scale whether each of 20 statements applies to him/her.

The Brief Symptom Inventory (Derogatis, (1993) BSI Brief Symptom Inventory. Administration, Scoring and Procedures Manual (4^{th} Ed). Minneapolis, Minn.: National Computer Systems) consists of 53 questions covering nine symptom dimensions: Obsession-Compulsion, Interpersonal Sensitivity, Depression, Anxiety, Hostility, Phobic anxiety, Paranoid ideation and Psychoticism (each rated on a 0-4 scale). Only the subset of questions used to measure anxiety are used (items 1, 12, 19, 38, 45, 49). Subjects rate themselves on these questions which should require no more than 2-3 min

The Liebowitz Social Anxiety Scale (Liebowitz, Mod. Prob. Pharmacopsychiatry 22: 141-173 (1987)) is a questionnaire that lists 24 social situations on which the subject rates (0-3) how much fear/anxiety he/she would experience, and how much he/she would avoid each situation (0-3).

### Oxytocin Assay:

Plasma OT concentrations are assayed using a standard EIA (Holt-Lunstad, Psychosom. Med. 70: 976-985 (2008)).

### Example 10

### Oxytocin Treatment of Social Deficits and Other Psychotic Symptoms in Schizophrenia (6 Week Treatment Trial)

### Purpose:

Compare the effects of 6 weeks of daily intranasal administration of oxytocin vs. placebo on social cognition, paranoia and other psychotic symptoms, and social competence in patients with schizophrenia.

### Social Cognition Hypotheses:

Oxytocin treatment is superior to placebo in improving social cognition in three domains: emotion recognition, attributional style and theory of mind.

### Paranoia Hypothesis:

Oxytocin treatment is superior to placebo in reducing paranoia and increasing trust.

### Psychotic Symptoms Hypothesis:

Oxytocin treatment is superior to placebo in improving global psychotic symptoms (e.g., lowering total PANSS scores).

### Exploratory Social Competence Question:

Is oxytocin treatment superior to placebo in improving social competence?

### Rationale:

Social dysfunction is among the most common symptoms in schizophrenia as well as the greatest cause of disability (Pinkham et al. (2003) Am. Psychiatry 160: 815-824; Yager & Ehman, Psychiatry 69: 47-68 (2006)) and one of the most treatment-resistant features of this disorder (Buchanan et al. Am J. Psychiatry 155: 751-760 (1998); Kirkpatrick et al., Neuropsychopharmacology 22: 303-310 (2000); Bellack et al., Beh. Therapy 37: 339-352 (2004)). Research has shown that social dysfunction is related to deficits in some domains of social cognition; emotion recognition, attributional style and theory of mind (Phillips et al., Biol. Psychiatry 54: 515-528 (2003); Pinkham et al. (2003) Am. J. Psychiatry 160: 815-824; Yager & Ehman, Psychiatry 69: 47-68 (2006)). Paranoia about other people's intentions also impairs social function (Gay & Combs, Schiz. Res. 80: 361-362 (2005)).

Our hypothesis is that intranasal administration of oxytocin in individuals with schizophrenia can improve social cognition and social motivation as well as decrease paranoia thereby improving social functioning.

### Subjects:

Eighty subjects, 18-55 years of age, who have had DSM-IV schizophrenia, paranoid or undifferentiated type, for at least one year. There is no selection on the basis of gender, ethnicity or race.

### Inclusion/Exclusion Criteria:

### Inclusion Criteria:

meeting DSM-IV criteria for paranoid or undifferentiated schizophrenia for at least 1 year; scoring ≧4 on the suspiciousness/persecution (hereafter referred to as paranoia) subscale of the Positive and Negative Symptoms Scale (PANSS) or 3 on the paranoia subscale and ≧3 on the hostility, active social avoidance, passive/apathetic social withdrawal or uncooperativeness subscale and ≧60 on the full PANSS: stability of symptom severity and on the same medication(s) and dose(s) for at least 1 month; low to moderate depressive symptoms.

### Exclusion Criteria:

Low literacy as indicated by an inability to read and understand the consent form; dependence on substances other than tobacco or caffeine; positive urine drug screen for illegal substances or drugs that have not been prescribed; debilitating medical conditions (including AIDS; HIV infection alone will not be grounds for exclusion); major surgery or trauma in the past year will be grounds for exclusion although subjects determined to be recovered and stable may be included at the discretion of the PI; pregnancy, breast-feeding; having given birth in the past 6 months or breast-feeding in the past 3 months. Individuals judged unable to learn self-administration of intranasal treatments and/or not sufficiently reliable to do so will be excluded. Abnormalities found during medical evaluation will be grounds for exclusion although subjects with laboratory measures somewhat out of the normal range may be included at the discretion of the PI.

### Study Design:

We will use a randomized, double-blind, placebo-controlled design. 80 subjects will complete the protocol; 40 subjects will be treated with oxytocin and 40 with normal saline placebo. Prior to the treatment trial, baseline measures of social cognition, social competence, social functioning, paranoia, other psychotic and psychiatric symptoms will be obtained. Social cognition and psychiatric symptoms will be measured on days 15, 29 and 43 of the 6 week treatment trial. Social competence measures will be obtained on day 15 and 43 of the treatment trial and social function will be measured again on day 43.

### Procedures:

Subjects are recruited from the University of North Carolina (UNC) Department of Psychiatry Schizophrenia Treatment and Evaluation Program outpatient clinics, the UNC Hospitals Psychotic Disorders inpatient unit, the inpatient units of Dorothea Dix Hospital (Raleigh, N.C.), schizophrenic patients who have completed 3 other studies (The Social Cognition and Interaction [SCIT] Randomized Control Trial [RCT]; Loving Kindness Meditation Study; The Genomic Psychiatry Cohort Study of Schizophrenia), and from the community. After giving informed consent, they are evaluated using the Positive and Negative Symptoms Scale (PANSS). If their total PANSS score is ≧60, their PANSS sub-score for paranoia is ≧4 (maximum sub-score is 7) or 3 on the paranoia subscale and ≧3 on the hostility, active social avoidance, passive/apathetic social withdrawal or uncooperativeness subscale, their medical history is reviewed including medications and dosages over the past month. If they do not have exclusionary medical or medication history, their psychiatric history is reviewed. If necessary to confirm their diagnosis, they undergo the Structured Clinical Interview for DSM-IV (SCID) patient version. If they have met criteria for schizophrenia, paranoid or undifferentiated type, for at least one year, they undergo a physical exam and blood is drawn and urine collected for laboratory measures including CBC with differential, electrolytes, BUN, creatinine, liver function tests, glucose, TSH, urinanalysis, urine drug screen and pregnancy test. This initial evaluation may require up to 3-4 hours. Prospective subjects are encouraged to rest for 20-30 minutes between the initial interviews (PANSS, SCID) and the history, physical exam and blood/urine collections. Psychiatric, medical and medication history is confirmed by reviewing medical records and contacting clinicians in charge of the prospective subject's psychiatric care. The initial evaluation is conducted in the Clinical and Translational Research Center (CTRC) or at the UNC Psychiatry Clinical Research Unit (CRU) at Dorothea Dix Hospital.

SCID, PANSS, other psychological results and medical labs and history are reviewed during conferences with Dr. Pedersen accompanied by Drs. Gilmore, Salimi and/or Rau. Drs. Pedersen and Gilmore are Board-certified Psychiatrists and Full Professors in the UNC Department of Psychiatry. Drs. Salimi and Rau are Board-certified Assistant Professors in the UNC Department of Psychiatry and co-Directors of the Department of Psychiatry CRU at Dorothea Dix Hospital. Consensus decisions are made during these conferences about whether subjects meet the diagnostic and other criteria for inclusion in the study.

If prospective subjects meet inclusion but not exclusion criteria, they are eligible to continue in the study. In the week prior to initiating the daily treatment trial, baseline measurements are obtained by administering all social cognition, social competence and social functioning tests as well as psychiatric rating instruments described below. This requires approximately 3-4 hours. Social cognition and competence testing are conducted by Psychology graduate or advanced undergraduate students or postdoctoral fellows who have been trained and are supervised by Dr. Penn, Professor of Clinical Psychology and an expert in social cognition measurement and a prominent investigator of social cognition deficits in psychotic disorders and autism. Psychiatric ratings are made by one of the Co-PIs, Psychology graduate students, an experienced research RN or a Psychiatry resident. Research staff conducting social cognition testing and psychiatric ratings are blind to treatment group. Baseline measurements are conducted in the CTRC outpatient clinic or at the UNC Psychiatry Clinical Research Unit (CRU) at Dorothea Dix Hospital.

During the treatment trial, twice daily test treatments are self-administered by subjects in the morning shortly before or after breakfast and late in the afternoon/early evening prior to dinner. Each treatment consists of six 0.1 ml insufflations (3/nostril alternating between nostrils) of SYNTOCINON® Spray (Novartis), which contains approximately 24 international units (IU) of oxytocin, or placebo. There is a 30-60 second pause between each insufflation. The placebo is a solution formulated by Triangle Compounding Pharmacy that has the same ingredients as SYNTOCINON® Spray except for oxytocin. Oxytocin and placebo test solutions are packaged as 30 ml in identical 60 ml spray vials designed to deliver 0.1 ml metered volume per insufflation. Treatment assignments are random within each gender. Vials containing oxytocin and placebo spray are relabeled by the Dorothea Dix Hospital Pharmacy or UNC Investigational Drug Service so that subjects and raters are blind to treatment. Social cognition testing begins 50 min after the morning treatment on days 15, 29 and 43 of the treatment trial and is followed by ratings of psychotic, mood and anxiety symptoms (requiring 1-3 hr depending on how much social cognition testing is done). Outpatient subjects are instructed to not take their AM dose of test substance before coming to their clinic visit on these days. They take that dose under observation of the CTRC or CRU nurses so that blood draws and testing can be performed at set times after the dose is given. The ER-40, Reading the Mind in the Eyes test (Eyes Test), IRI, and Trustworthiness Tasks (see below) as well as psychiatric ratings are conducted at all time points. The BACS, Brüne, and AIHQ are conducted at baseline, Day 15 and Day 43. The social competence role plays and the SLOF questionnaire are completed at baseline and day 43. The Informant version of the SLOF is conducted via telephone; the study personnel ask the informant basic screening questions to assess if they know the subject well and then ask the SLOF questions. On these treatment days, subjects take 20-30 min breaks after the ER-40, Eyes Test, Trustworthiness and BACS tasks and after the ensuing Brüne, AIHQ, and social competence tasks which are followed by psychiatric ratings. At the end of the treatment protocol (treatment day 43), subjects and research staff who have conducted social cognition testing or made psychiatric ratings are asked to guess which treatment the subjects received.

Subjects may take part in this study as outpatients or inpatients or may start as inpatients and complete the protocol in the outpatient setting. Subjects are trained by research nurses in intranasal self-administration of test treatments after the baseline assessment is complete and the prospective subject appears to meet criteria (screening lab values will not yet be available at this time point). All 10 subjects who completed the 2 wk preliminary study at the Dorothea Dix CRU, quickly and easily mastered this procedure. After baseline assessments are complete, all subjects take their first intranasal dose of test substance while being observed by nurses. Subjects who wish to participate in the study as outpatients are given written instructions about the timing of each day's test treatments and other procedures to follow at home. They are then given a 60 ml spray vial containing 30 ml of test substance and other supplies to last until their first outpatient clinic testing appointment. Subjects who wish to be studied in the hospital who are not already inpatients on the CRU are admitted shortly after completing baseline measurements. Outpatient subjects visit an outpatient study site (CTRC or CRU) on the mornings of treatment days 15, 29, and 43 (where they are provided with a snack or breakfast and lunch depending on the length of the visit). They self administer test substance at the site so staff can confirm they are using proper technique. Outpatient and inpatient subjects fast after midnight the night before baseline and treatment days 15 and 43 assessment clinic visits. Prior to breakfast on the morning of the baseline assessment, blood (10 ml) is drawn to obtain serum that is frozen to measure lipid panel, glucose, ALT, AST, alkaline phosphatase and total bilirubin. Prior to breakfast on treatment days 15 and 43, blood (20 ml) is drawn for lab values that are measured immediately (CBC with differential, glucose, Na⁺, K⁺, GGT, BUN, creatinine) and to obtain serum to be frozen for later measurement of lipid panel, ALT, AST, alkaline phosphatase and total bilirubin. Urine is also collected before breakfast during the baseline and treatment days 15 and 43 visits and frozen for later measurement of osmolality. Some of the urine collected on treatment day 43 is used to immediately obtain a UA and drug screen. At 40 min after the last intranasal insufflation on treatment days 15, 29 and 43, blood (7.5-15 ml) is drawn to measure plasma oxytocin concentrations and (at treatment day 29) Na, K and non-fasting glucose. Vital signs are obtained approximately 3-4 hr after intranasal treatment (when social cognition/competence testing and psychiatric ratings are completed). An ECG is also obtained on the morning of treatment days 15 and 43 immediately after vital sign measurements. Urine is also collected from female subjects on the day of baseline measurements and on treatment days 15, 29 and 43 for pregnancy testing. These same procedures are conducted on the morning of baseline and treatment days 15, 29 and 43 assessments in subjects studied as inpatients. Screening visit labs are repeated at baseline if the baseline visit doesn't occur within 10 days of the screening visit.

After procedures at the study site on the morning of treatment days 15 and 29, outpatient subjects are given another 60 ml spray vial containing 30 ml of test solution. Each vial is used to administer twice daily doses for 14 days. Subjects are instructed to store vials at room temperature and to clean the tip of the spray nozzle with an alcohol wipe after each dose is administered. There are 2 checks on whether outpatient subjects are complying with self-administration of oxytocin. First, subjects return the vial to research staff at each visit to the outpatient study site. Secondly, nurses weigh each new vial before the subject takes it home and after the subject takes their morning dose at the next clinic visit. Subjects are dropped from the study if they don't return to the clinic with the vials given to them at baseline and treatment day 15 on treatment day 29. In addition, if on treatment days 15 and 29 the decline in the weight of the vial is less than 75% of the expected 21 g (indicating complete compliance), the subject may be dropped from the study. With the participant's permission, our research staff give outpatient participants brief daily reminder calls on weekday mornings to take the study treatment.

### Measurements:

A battery of well-validated instruments to quantify psychiatric symptoms, social cognition and social competence are used in this study. The domains to be measured and the instruments that will be used are summarized below. Psychiatric symptoms will be rated by research staff among which good inter-rater reliability will be established.

These instruments not discussed in detail below are described further in Example 9.

### Psychiatric Measures:

The Positive and Negative Symptoms Scale (PANSS)
Depressive symptoms: Beck Depression Inventory: The Beck Depression Inventory (BDI) is a 21 item instrument that assesses cognitive, affective and somatic symptoms of depression (Beck et al, Arch. Gen Psychiatry 4: 561-569 (1961)). The BDI has acceptable test-retest reliability (r=0.79) in non-clinical populations and demonstrates concurrent validity (range from 0.67 to 0.79) in both clinical and non-clinical populations.
Anxiety (general and social) and other psychiatric symptoms: The Brief Symptom Inventory (BSI) and The Liebowitz Social Anxiety Scale
Paranoia: The Paranoia Scale.

### Social Cognition Measures:

The ER-40
The Reading the Mind in the Eyes Test—Revised Version
Trustworthiness task
The Theory of Mind Picture Stories Task
The Ambiguous Intentions Hostility Questionnaire (AIHQ)
Interpersonal Reactivity Index (IRI).
Brief Assessment of Cognition in Schizophrenia (BACS)

### Social Skill Measures:

Social competence is assessed by two brief, 90-sec role plays (which are recorded). The first role-play is an unstructured conversation in which the research confederate plays the role of a new neighbor with whom the subject is instructed to strike up a conversation. In the second role-play, the research confederate plays an upset friend who the subject is instructed to attempt to console.
Specific Levels of Functioning Scale (SLOF)

### Other Measures:

Food Cravings and Night Eating Questionnaires. Atypical antipsychotic medications, (which most subjects will be taking), increase appetite and may contribute to weight gain. Oxytocin is known to affect food intake in animals. Therefore, these measures are included that have been used in previous studies of weight gain/appetite change in patients taking atypical antipsychotic medications.
   Source of Oxytocin Nasal Spray and Rationale for Using this Form of Oxytocin:

Novartis, the manufacturer of oxytocin nasal spray (SYNTOCINON® Spray) took this product off the US market (it had been FDA-approved since 1962) because of poor sales in the US, not because of safety concerns. SYNTOCINON® Spray remains commercially available by prescription in Europe and can be easily obtained from international pharmacies. The intravenous and intramuscular injectable form of oxytocin that is FDA approved and marketed in the US is not a suitable substitute because it does not have the ingredients added to the SYNTOCINON® Spray that promote absorption across the nasal epithelium.

### Results:

Accrual of patients into this trial has commenced and is ongoing. Six-week data from the trial described in Example 9 can be combined with the results from this study to produce a 6-week treatment effect report.

### Example 11

### Oxytocin Treatment of Other Psychotic Disorders or Psychotic Symptoms in Mood Disorders

Diagnostic criteria for schizoaffective disorder and delusional disorder as well as major depressive episodes and manic episodes with psychotic features are summarized in the DSM-IV-R. Subjects with these disorders are given oxytocin treatment (e.g., intranasally) to treat psychotic symptoms. For example, the effectiveness of twice daily intranasal administration of oxytocin (24 international units per dose) is compared with placebo for treating one of these disorders in a double-blind trial in which psychotic symptoms are quantified using well-established rating instruments.

### Example 12

### Oxytocin Treatment of Disorders Characterized by Anxiety, Fear, Depression, Pain and Intolerance of Stress

Diagnostic criteria for posttraumatic stress disorder and premenstrual dysphoric disorder are summarized in the DSM-IV-R. Posttraumatic stress disorder symptoms develop after the affected individual experiences, witnesses or is confronted with an event or events that involved actual or threatened death or serious injury, or a threat to the physical integrity of self or other to which the individual responded with intense fear, helplessness or horror. Firm diagnostic criteria for fibromyalgia have not yet been established so this condition is viewed as a syndrome rather than a disorder. Diagnosis requires ruling out other medical disorders that may be causing the symptoms. The hallmark symptoms are chronic muscle and joint tenderness and pain that often shift location in the body. These symptoms are frequently accompanied by fatigue and depression. Subjects with any one or more of these disorders are given oxytocin treatment (e.g., intranasally) to treat symptoms of these disorders. For example, the effectiveness of twice daily intranasal administration of oxytocin (24 international units per dose) is compared with placebo for treating one of these disorders in a double-blind trial in which symptoms of these disorders are quantified using well-established rating instruments. Further, subjects experiencing a severely traumatizing event or events can be treated with oxytocin shortly thereafter to prevent the subsequent development of posttraumatic stress disorder The effectiveness of administration of multiple doses of intranasal oxytocin (24 international units per dose) is compared with administration of the same number of doses of placebo in a double-blind trial and following subjects over time and comparing the incidence of posttraumatic stress disorder in the treatment groups over various periods of time following the trauma exposure.

### Example 13

### Oxytocin Treatment to Reduce the Risk of Addiction in Subjects Treated with Opioids for Pain Relief

Subjects that are treated with opioids for pain relief face the risk of developing opioid dependence (it is difficult to determine a priori which subjects are addiction prone). Further, as opioid tolerance develops, increasing dosages of opioids may have to be administered to maintain efficacy. In addition, if dependence does develop, the subject can experience withdrawal symptoms once opioid treatment is stopped.

Administration of oxytocin in subjects administered opioids for pain relief can reduce the likelihood of dependence, tolerance (with a resulting need for increasing dosages to control pain), reduce the total amount of opioid administered, avoid the need to increase the dosage of the opioid to maintain pain relief, allow a more rapid tampering off of the opioid, and reduce withdrawal symptoms after cessation of the opioid (if dependence develops).

In the first study, the subjects are mammalian subjects (e.g., human subjects) receiving opioid treatment for pain relief. Optionally, only patients receiving opioid treatment for pain control for a prolonged period of time (e.g., at least about 1, 2, 3, 4 or 6 weeks or 2, 3, 4, 6, 9 or 12 months or longer) are included.

In a subsequent study, the subjects are mammalian subjects (e.g., human subjects) receiving opioid treatment for pain relief; however, in this study, the subjects are currently addicted to a substance (e.g., alcohol, cocaine and/or any other addictive substance as described herein) or have a history of such addiction. These subjects may be particularly at high risk for developing opioid addiction and/or tolerance. Minimum time limits for opioid administration can be established as discussed above.

In a further study, the subjects are mammalian subjects (e.g., human subjects) that are not currently addicted to a substance and do not have a history of such addiction. Minimum time limits for opioid administration can be established as discussed above.

Subjects being administered opioids for pain relief are given oxytocin treatment (e.g., intranasally) to reduce the likelihood of opioid dependence, opioid tolerance and/or to reduce withdrawal symptoms. For example, the effectiveness of two or more daily intranasal administrations of oxytocin (24 international units per dose) is compared with placebo in which opioid dependence, opioid tolerance and/or withdrawal symptoms are evaluated using well-established rating instruments. Further, pain scores are evaluated to determine whether oxytocin treatment has any effect on opioid induced pain relief (e.g., enhancing or reducing pain relief). Oxytocin can further be administered before and/or after opioid treatment. For example, oxytocin can be administered following the end of opioid treatment to reduce withdrawal symptoms.

Research end points are (1) how much opioid is needed for pain control as compared with placebo-treated patients, (2) how well pain is controlled; (3) how rapidly opioid can be tapered off, and (4) how much withdrawal patients experience.

The foregoing is illustrative of the present invention, and is not to be taken as limiting thereof. The invention is defined by the following claims, with equivalents of the claims to be included therein.

1. A method of treating a psychiatric or medical disorder in a human subject in need thereof, the method comprising administering to the subject an effective amount of an oxytocin receptor agonist.

2. The method of claim 1, wherein the method comprises intranasal administration of the oxytocin receptor agonist.

3. The method of claim 1, wherein the disorder is a dependence disorder.

4. (canceled)

5. The method of claim 4, wherein the dependence disorder is a substance use disorder of alcohol dependence, opioid dependence, nicotine dependence, cocaine dependence, cannabis dependence, benzodiazepine dependence, gambling disorder and/or sexual addiction.

6. (canceled)

7. The method of claim 1, wherein the disorder is a psychotic disorder or a mood disorder with psychotic features.

8. The method of claim 7, wherein the psychotic or mood disorder is schizophrenia, schizoaffective disorder, delusional disorder, a depressive disorder with psychotic features, and/or a bipolar disorder with psychotic features.

9. The method of claim 8, wherein the method is practiced to reduce a psychotic symptom, increase social cognition, increase social functioning, increase empathy, reduce paranoia, increase trust of others and/or reduce hostility.

10. The method of claim 1, wherein the disorder is a medical disorder characterized by anxiety, fear, depression, pain and/or intolerance to stress.

11. (canceled)

12. The method of claim 10, wherein the method is practiced to reduce anxiety, fear, depression, pain and/or intolerance to stress.

13. The method of claim 1, wherein the disorder is a disorder characterized by social dysfunction and/or lack of empathy.

14. (canceled)

15. The method of claim 13, wherein the method is practiced to increase social function, social cognition and/or empathy.

16. The method of claim 1, wherein the subject is in the prodromal period of a psychotic disorder.

17. The method of claim 1, wherein the subject is experiencing the first episode of a psychotic disorder and/or a mood disorder with psychotic features.

18-19. (canceled)

20. The method of claim 1, wherein the oxytocin receptor agonist is oxytocin.

21. (canceled)

22. A pharmaceutical composition comprising an effective amount of an oxytocin receptor agonist and a pharmaceutically acceptable carrier for the treatment of a psychiatric disorder or a medical disorder in a human subject and/or for increasing social cognition and/or social functioning in a human subject and/or for preventing opioid dependence, opioid tolerance and/or opioid withdrawal symptoms in a mammalian subject receiving opioid treatment for pain relief.

23. The pharmaceutical composition of claim 22, wherein the pharmaceutical composition is formulated for intranasal administration.

24-33. (canceled)

34. A method of preventing opioid dependence, opioid tolerance and/or opioid withdrawal symptoms in a mammalian subject receiving opioid treatment for pain relief, the method comprising administering to the subject an effective amount of an oxytocin receptor agonist.

35. The method of claim 34, wherein the subject is currently dependent on an addictive substance or has a history of such addiction.

36. The method of claim 34, wherein the subject is not currently dependent on an addictive substance or have a history of such addiction.

37. The method of claim 34, wherein the oxytocin receptor agonist is oxytocin.

38-39. (canceled)
